(19) United States
(12) Patent Application Publication
(10) Pub. No.: US 2024/0226464 A1
(43) Pub. Date: Jul. 11, 2024

(54) DRUG DELIVERY ADJUSTMENT

(71) Applicant: JANSSEN PHARMACEUTICALS, INC., Titusville, NJ (US)

(72) Inventors: Francesco N. ALBERTINI, Pleasanton, CA (US); Gregory J. BAKOS, Mason, OH (US); Michael A. BARATTA, West Chester, PA (US); Michael CANNAMELA, Atlantic Highland, NJ (US); Anthony R. DIUBALDI, Jackson, NJ (US); Yueheng DOU, Fall River, MA (US); Wayne DREVETS, Rancho Santa Fe, CA (US); James A. FLEMING, Bethlehem, PA (US); Jason L. HARRIS, Lebanon, OH (US); Kevin L. HOUSER, Navarre, FL (US); Emma Louise HUBERT, San Jose, CA (US); Michael HUTCHINSON, King of Prussia, PA (US); Monica A. KAPIL, San Jose, CA (US); Peter KRULEVITCH, Pleasanton, CA (US); Chaley John LARSON, Pottstown, PA (US); Wouter Jacques Noel LEQUIEU, Lievegem (BE); Kui LIU, IV, Redwood City, CA (US); Frederick E. SHELTON, IV, Cincinnati, OH (US); George SZABO, Hatfield, PA (US); Steven M. VESOLE, San Bruno, CA (US); Jingli WANG, San Jose, CA (US); Hong YAN, Livermore, CA (US)

(21) Appl. No.: 17/763,585

(22) PCT Filed: Sep. 24, 2020
(86) PCT No.: PCT/IB2020/058967

§ 371 Date: Nov. 20, 2023

Related U.S. Application Data
(60) Provisional application No. 62/905,446, filed on Sep. 25, 2019.
(60) Provisional application No. 62/905,448, filed on Sep. 25, 2019.
(60) Provisional application No. 62/905,451, filed on Sep. 25, 2019.
(60) Provisional application No. 63/020,931, filed on May 6, 2020.

(51) Int. Cl. A61M 15/00; G06T 7/00

(52) U.S. Cl. CPC A61M 15/0003; A61M 15/0066; G06T 7/00; A61M 2205/52; A61M 2230/201; A61M 2230/30; A61M 2230/50

(57) ABSTRACT

Provided herein are drug administration devices, methods, and systems for adjusting drug delivery to a patient to allow drug dosage to be adjusted based on a variety of different factors influencing the patient during administration of the drug. In one embodiment, a drug administration device or system can have a drug holder with a drug therein to be delivered to a patient, one or more sensors that are configured to gather various data associated with the patient, and at least one processor that can analyze the data gathered by the one or more sensors and adjust delivery of the drug based on the data.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Furthermore, consideration of surrounding circumstances of the patient during drug administration helps avoid adverse reactions caused by various factors beyond just an initial dose of a drug, but it can be difficult to assess these circumstances at all or in a timely manner. Safe drug administration and personalized patient care may thus be adversely affected.

In relation to how a drug is administered to the patient, there are various dosage forms that can be used. For example, these dosage forms may include parenteral, inhalational, oral, ophthalmic, nasal, topical, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors, jet injectors, and infusion pumps), nasal spray devices, and inhalers.

It can be desirable to monitor compliance with the guidance that is associated with the drugs that are administered to a patient in various dosage forms. This can provide assurance that correct procedures are being followed and avoid the adoption of incorrect and potentially dangerous approaches. Further, this can also enable optimization of the administration of the drug to the patient.

### SUMMARY

In general, devices, methods, and systems are provided herein for drug delivery adjustment. The devices, methods, and systems may allow for adjustment of drug dosages based on one or more surrounding circumstances of the patient during drug administration.

In one aspect, a drug administration device is provided that in one embodiment includes a drug holder configured to retain a drug therein. The device also includes a first sensor configured to gather data regarding a first characteristic associated with a patient, a second sensor configured to gather data regarding a second characteristic associated with the patient, a memory configured to store therein an algorithm including at least one variable parameter, and a processor. The processor is configured to control delivery of a first dose of the drug from the drug holder to the patient by executing the algorithm, change the at least one variable parameter of the algorithm stored in the memory based on the data gathered by the first sensor and data gathered by the second sensor, and after changing the at least one variable parameter, control delivery of a second dose of the drug from the drug holder to the patient by executing the algorithm.

The device can have any number of variations. For example, the processor can also be configured to automatically control delivery of the doses according to a predetermined schedule of dosing for the patient. In another example, the device can include at least one additional sensor, each sensor can be configured to gather data regarding a different characteristic, and the processor can be configured to change the at least one variable parameter of the algorithm stored in the memory based on the data gathered by the at least one additional sensor. In another example, the processor can be further configured to consider the data gathered by each of the first and second sensors in a hierarchy in changing the at least one variable parameter. In still another example, the first characteristic can be a physiological characteristic of the patient, and the second characteristic can be a situational characteristic of the patient. In another example, the first characteristic can be one of blood sugar level, blood pressure, perspiration level, and heart rate, and the second characteristic can be at least one of core temperature, tremor detection, time of day, date, patient activity level, blood pressure, metabolic rate, altitude, temperature of the drug, viscosity of the drug, GPS information, angular rate, current of a motor used in delivering the drug, blood oxygenation level, sun exposure, osmolality, and air quality. In still another example, the second sensor can be configured to gather data by capturing an image of at least one of the patient and an environment in which the patient is located, and the processor can be configured to analyze the image to determine at least one of whether food intake occurred and skin reaction to the drug. In yet another example, the processor of the drug administration device, e.g., an injection device, a nasal spray device, and an inhaler, can also be configured to, based on at least one of the data gathered by the first sensor and the data gathered by the second sensor, cause a device operation prevention mechanism to move from an unlocked state, in which the device operation prevention mechanism allows delivery of the drug to a user, to a locked state, in which the device operation prevention mechanism prevents delivery of the drug to the user. In another example, the drug can include a biologic, and the second characteristic can be an inflammatory response. In yet another example, the drug can include insulin, and the first characteristic can be blood sugar level. In still another example, the drug can include glucagon, and the first characteristic can be blood sugar level. In another example, the drug can include a blood pressure medication, and the first characteristic can be blood pressure. In another example, the at least one variable parameter can include a rate of delivery of the drug from the drug holder to the patient. In still another example, the at least one variable parameter can include a time interval between dose deliveries such that doses delivered after the changing of the at least one variable parameter are at a different time interval than doses delivered before the changing of the at least one variable parameter. In another example, changing the at least one variable parameter can result in the processor controlling delivery of the second dose such that the second dose is not delivered to the patient. In yet another example, the processor can be configured to automatically change the at least one variable parameter. In another example, the processor can also be configured to cause a notification to be provided to the patient based on the data gathered by the second sensor.

In another example, the device also can include a communications interface configured to wirelessly transmit data indicative of the data gathered by the first sensor and data gathered by the second sensor to a remotely located computer system, and, in response, to wirelessly receive a command from the remotely located computer, and the processor can be configured to change the at least one variable parameter only after the communications interface receives the command.

In yet another example, the processor can be configured to change the at least one variable parameter of the algorithm during the delivery of the second dose such that the algorithm is changed in real time with the delivery of the second dose. In another example, the processor can be configured to change the at least one variable parameter of the algorithm before a start of the delivery of the second dose.

For still another example, the memory can also be configured to store therein manually input data regarding the patient, and the processor can also be configured to change the at least one variable parameter of the algorithm stored in the memory based on the stored input data. For another example, the drug can include at least one of infliximab, golimumab, ustekinumab, daratumumab, guselkumab, epoetin alfa, risperidone, esketamine, ketamine, and paliperidone palmitate.

In another embodiment, a drug administration device is provided that includes a drug holder configured to retain a drug therein, a first sensor configured to gather data regarding a physiological characteristic of a patient, a second sensor configured to gather data regarding a physical characteristic of the patient, a memory configured to store therein an algorithm including at least one variable parameter, and a processor. The processor is configured to control delivery of a first dose of the drug from the drug holder to the patient by executing the algorithm, change the at least one variable parameter of the algorithm stored in the memory based on the data gathered by the first sensor and data gathered by the second sensor, and after changing the at least one variable parameter, control delivery of a second dose of the drug from the drug holder to the patient by executing the algorithm.

The device can have any number of variations. For example, the processor can also be configured to automatically control delivery of the doses according to a predetermined schedule of dosing for the patient. In another example, changing the at least one variable parameter can compensate for the physical characteristic. In another example, the physical characteristic can be one of temperature, metabolic demand, and cognitive function. In another example, the physiological characteristic can be at least one of body temperature, and heart rate, and the physical characteristic can be metabolic demand measured using at least one of food intake and BMR (basal metabolic rate). In still another example, the physical characteristic can be weight. In yet another example, the processor can be configured to change the at least one variable parameter of the algorithm during the delivery of the second dose such that the algorithm is changed in real time with the delivery of the second dose. In another example, the processor can be configured to change the at least one variable parameter of the algorithm before a start of the delivery of the second dose. For still another example, the memory can also be configured to store therein manually input data regarding the patient, and the processor can also be configured to change the at least one variable parameter of the algorithm stored in the memory based on the stored input data. For another example, the drug can include at least one of infliximab, golimumab, ustekinumab, daratumumab, guselkumab, epoetin alfa, risperidone, esketamine, ketamine, and paliperidone palmitate.

In another embodiment, a drug administration device is provided that includes an autoinjector that includes a drug holder configured to retain a drug therein, a plurality of sensors configured to gather data regarding an angular orientation of the autoinjector relative to skin of a patient, a memory configured to store therein an algorithm including at least one variable parameter, and a processor. The processor is configured to control delivery of a dose of the drug from the drug holder to the patient by executing the algorithm, change the at least one variable parameter of the algorithm stored in the memory based on the data gathered by the plurality of sensors.

The device can have any number of variations. For example, the processor can be configured to change the at least one variable parameter of the algorithm to prevent delivery of the drug from the autoinjector in response to the gathered data indicating that the autoinjector is not at a substantially perpendicular angle relative to the skin of the patient, and the processor can be configured to change the at least one variable parameter of the algorithm to allow delivery of the drug from the autoinjector in response to the gathered data indicating that the autoinjector is at the substantially perpendicular angle relative to the skin of the patient. For another example, the autoinjector can also include a trigger configured to be actuated to cause delivery of the drug from the drug holder to the patient, and the at least one variable parameter of the algorithm can represent whether or not the trigger is able to be user-actuated to cause the delivery of the drug. For yet another example, the autoinjector can also include a device operation prevention mechanism configured to move between a locked state, in which the device operation prevention mechanism prevents delivery of the drug from the autoinjector, and an unlocked state, in which the device operation prevention mechanism allows delivery of the drug from the autoinjector, and the processor can be configured to cause the device operation prevention mechanism to move from the locked state to the unlocked state in response to the gathered data indicating that the autoinjector is at a substantially perpendicular angle relative to skin of a patient. For still another example, the processor can be configured to change the at least one variable parameter of the algorithm before a start of the delivery of the dose. For another example, the plurality of sensors can include contact sensors. For still another example, the plurality of sensors can include pressure sensors. For another example, the drug can include at least one of infliximab, golimumab, ustekinumab, daratumumab, guselkumab, epoetin alfa, risperidone, esketamine, ketamine, and paliperidone palmitate.

In still another embodiment, a drug administration system is provided that in one embodiment includes a drug administration device and an accessory. The drug administration device is configured to retain a drug therein for delivery to a patient and includes a sensor configured to gather data regarding a physiological characteristic of the patient. The accessory includes a processor that is configured to receive data from the sensor indicative of the gathered data and to control delivery of the drug to the patient based on the received data.

The system can have any number of variations. For example, the accessory and the drug administration device can be separate devices. In at least some embodiments, the accessory can be configured to be worn by the patient and can include one of an ear piece, a smart watch, a fingernail sensor, a digital collection patch, augmented reality glasses, and a camera. In at least some embodiments, the accessory can be configured to be implanted in or ingested by the patient. In at least some embodiments, the accessory can be configured to gather data by capturing an image of at least one of the patient and an environment in which the patient is located, and the processor can also be configured to analyze the image to determine at least one of whether food intake occurred and skin reaction to the drug.

In another example, controlling the delivery can include adjusting at least one of a dosage of the drug, a timing between doses of the drug, and a location of delivery of the drug. In another example, the physiological characteristic can include a reaction of the patient to the drug delivered thereto. In still another example, the physiological characteristic can include at least one of angular rate, blood oxygenation level, sun exposure, and osmolality. In another example, the sensor can include a biosensor configured to sense an enzyme, an antibody, a histamine, or a nucleic acid. In another example, the sensor can include a sensor array or a dual sensor. In another example, the drug can include insulin, and the physiological characteristic can be blood sugar level. In still another example, the drug can include glucagon, and the physiological characteristic can be blood sugar level. In yet another example, the drug can include a blood pressure medication, and the physiological characteristic can be blood pressure. For another example, the drug can include at least one of infliximab, golimumab, ustekinumab, daratumumab, guselkumab, epoetin alfa, risperidone, esketamine, ketamine, and paliperidone palmitate.

In another aspect, a drug administration method is provided that in one embodiment includes gathering data, using a first sensor, regarding a first characteristic associated with a patient. The method also includes gathering data, using a second sensor, regarding a second characteristic associated with the patient. The method also includes, with a processor, controlling delivery of a first dose of a drug from a drug administration device to the patient by executing an algorithm stored in a memory, changing at least one variable parameter of the algorithm stored in the memory based on the data gathered by the first sensor and data gathered by the second sensor, and after changing the at least one variable parameter, controlling delivery of a second dose from the drug administration device to the patient by executing the algorithm.

The method can have any of a variety of alterations. For example, the first characteristic can be a physiological characteristic of the patient, and the second characteristic can be a situational characteristic of the patient. In another example, the first characteristic can be one of blood sugar level, blood pressure, perspiration level, and heart rate, and the second characteristic can be at least one of core temperature, tremor detection, time of day, date, patient activity level, blood pressure, metabolic rate, altitude, temperature of the drug, viscosity of the drug, GPS information, angular rate, blood oxygenation level, sun exposure, osmolality, and air quality. In yet another example, the processor can change the at least one variable parameter of the algorithm during the delivery of the second dose such that the algorithm is changed in real time with the delivery of the second dose. In another example, the processor can change the at least one variable parameter of the algorithm before a start of the delivery of the second dose. In still another example, the memory can also have stored therein manually input data regarding the patient, and the processor can change the at least one variable parameter of the algorithm stored in the memory also based on the stored input data. For another example, the drug can include at least one of infliximab, golimumab, ustekinumab, daratumumab, guselkumab, epoetin alfa, risperidone, esketamine, ketamine, and paliperidone palmitate.

In another embodiment, a drug administration method is provided that includes gathering data, using a first sensor, regarding a physiological characteristic associated with a patient. The method also includes gathering data, using a second sensor, regarding a physical characteristic associated with the patient. The method further includes, with a processor, controlling delivery of a first dose of a drug from a drug administration device to the patient by executing an algorithm stored in a memory, changing at least one variable parameter of the algorithm stored in the memory based on the data gathered by the first sensor and data gathered by the second sensor, and after changing the at least one variable parameter, controlling delivery of a second dose from the drug administration device to the patient by executing the algorithm.

The method can vary in any number of ways. For example, the processor can automatically control delivery of the doses according to a predetermined schedule of dosing for the patient. In another example, changing the at least one variable parameter can compensate for the physical characteristic. In still another example, the physical characteristic can be one of temperature, metabolic demand, and cognitive function. In another example, the physiological characteristic can be at least one of body temperature, and heart rate, and the physical characteristic can be metabolic demand measured using at least one of food intake and BMR (basal metabolic rate). In still another example, the physical characteristic can be weight. In yet another example, the processor can change the at least one variable parameter of the algorithm during the delivery of the second dose such that the algorithm is changed in real time with the delivery of the second dose. In another example, the processor can change the at least one variable parameter of the algorithm before a start of the delivery of the second dose. In still another example, the memory can also have stored therein manually input data regarding the patient, and the processor can change the at least one variable parameter of the algorithm stored in the memory also based on the stored input data. For another example, the drug can include at least one of infliximab, golimumab, ustekinumab, daratumumab, guselkumab, epoetin alfa, risperidone, esketamine, ketamine, and paliperidone palmitate.

In another embodiment, a drug administration method is provided that includes gathering data, using a sensor of a drug administration device, regarding a physiological characteristic of a patient. The method also includes, with a processor of an accessory that is a separate device from the drug administration device, receiving data from the sensor indicative of the gathered data, and controlling delivery of the drug from the drug administration device to the patient based on the received data.

The method can vary in any number of ways. For example, the accessory can be worn by the patient and can include one of an ear piece, a smart watch, a fingernail sensor, a digital collection patch, augmented reality glasses, and a camera. In another example, the accessory can be implanted in or can be ingested by the patient. In another example, the accessory can gather data by capturing an image of at least one of the patient and an environment in which the patient is located, and the processor can analyze the image to determine at least one of whether food intake occurred and skin reaction to the drug. In still another example, controlling the delivery can include adjusting at least one of a dosage of the drug, a timing between doses of the drug, and a location of delivery of the drug. In still another example, the physiological characteristic can include a reaction of the patient to the drug delivered thereto. In another example, the physiological characteristic can include at least one of angular rate, blood oxygenation level, sun exposure, and osmolality. For another example, the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9 is a schematic view of another embodiment of a drug administration device;

FIG. 10 is a flow diagram of the drug administration device of FIG. 9 in use;

FIG. 11 is a graphical representation of the effects on a patient over time of another embodiment of a drug administration device in use;

FIG. 12 is a schematic view of another embodiment of a drug administration device;

FIG. 13 is a flow diagram of the drug administration device of FIG. 12 in use;

FIG. 14 is a perspective view of one embodiment of an accessory for use with a drug administration device on a patient in the form of an ear piece;

FIG. 15 is a perspective view of another embodiment of an accessory for use with a drug administration device on a patient in the form of a wrist band;

FIG. 16 is a perspective view of another embodiment of an accessory for use with a drug administration device on a patient in the form of a headband;

FIG. 17 is a perspective view of another embodiment of an accessory for use with a drug administration device attached to a patient's head;

FIG. 18 is a perspective view of another embodiment of an accessory for use with a drug administration device attached to a patient's abdomen;

FIG. 19 is a perspective view of another embodiment of an accessory for use with a drug administration device attached to a patient's back;

FIG. 20 is a perspective view of another embodiment of an accessory for use with a drug administration device attached to a patient's fingernail;

FIG. 21 is a perspective view of another embodiment of an accessory for use with a drug administration device attached to a patient's fingernail;

FIG. 22 is a partial cross-sectional view of another embodiment of an accessory for use with a drug administration device implanted in a patient;

FIG. 23 is a perspective view of another embodiment of an accessory for use with a drug administration device in the form of glasses that can view a patient's food;

FIG. 24 is a perspective view of another embodiment of an accessory for use with a drug administration device in the form of a smartphone photographing a patient;

FIG. 25 is a graphical representation of the patient's skin of FIG. 24 photographed over time and measured for a reaction;

FIG. 26 is a perspective view of the accessory of FIG. 24 photographing the patient;

FIG. 27 is a graphical representation of the patient's skin of FIG. 26 photographed over time and measured for a reaction;

FIG. 28 is a perspective view of another embodiment of an accessory for use with a drug administration device in the form of a smartphone photographing a patient;

FIG. 29 is a graphical representation of the patient's estimated body weight based on images of FIG. 28;

FIG. 30 is a perspective view of another embodiment of a drug administration device;

FIG. 31 illustrates various front views of a user interface of the device of FIG. 30 during a series of events;

FIG. 32 is a graphical representation of the effects on a patient over time of the drug administration device of FIG. 30 in use;

FIG. 33 is a graphical representation of the effects on a patient over time of another embodiment of a drug administration device in use;

FIG. 34 is a graphical representation of the effects on a patient over time of another embodiment of a drug administration device in use;

FIG. 35 is a front view of a user interface of another embodiment of a drug administration device;

FIG. 36 is a graphical representation of the effects on a patient over time of the drug administration device of FIG. 35 in use;

FIG. 37 is a side view of a distal portion of one embodiment of an autoinjector;

FIG. 38 is a distal end view of the autoinjector of FIG. 37;

FIG. 39 is a side view of the autoinjector of FIG. 37 in use;

FIG. 40 is a side view of a distal portion of the autoinjector of FIG. 37 not yet in contact with skin of a patient;

FIG. 41 is a side view of the distal portion of the autoinjector of FIG. 40 with the autoinjector in contact with the skin and at a proper angular orientation relative to the skin;

FIG. 42 is a side view of the distal portion of the autoinjector of FIG. 40 with the autoinjector in contact with the skin and at an improper angular orientation relative to the skin; and

FIG. 43 is a schematic view of one embodiment of a drug administration device and a powered add-on module.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

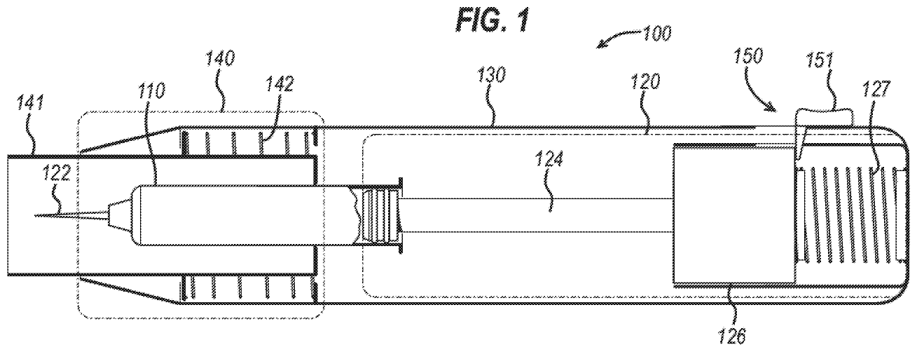

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

While the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

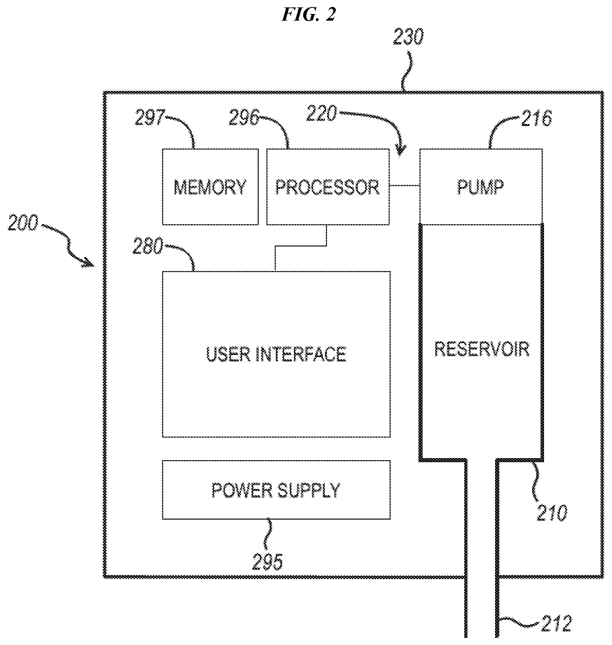

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

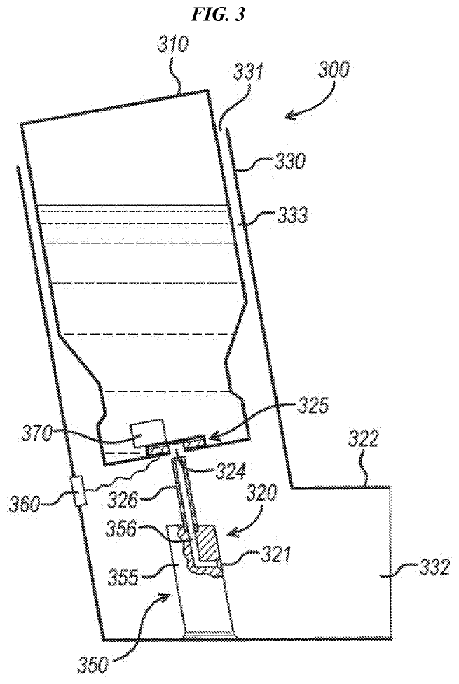

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Nasal Spray Device

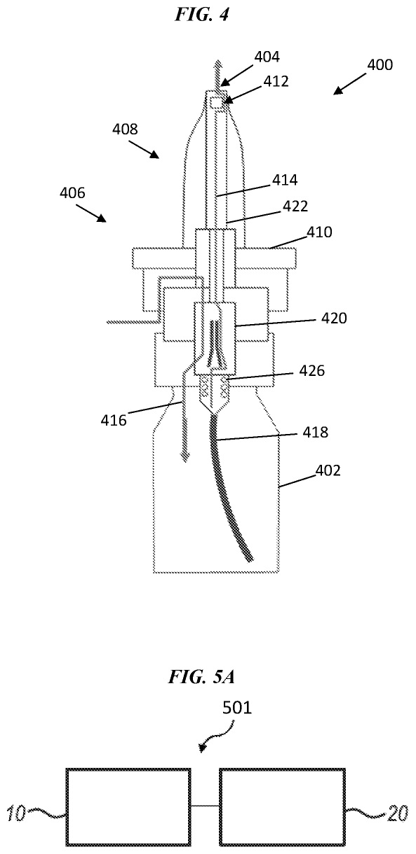

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-S®), naloxone (e.g., Narcan®), and sumatriptan (e.g., Imitrex®).

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

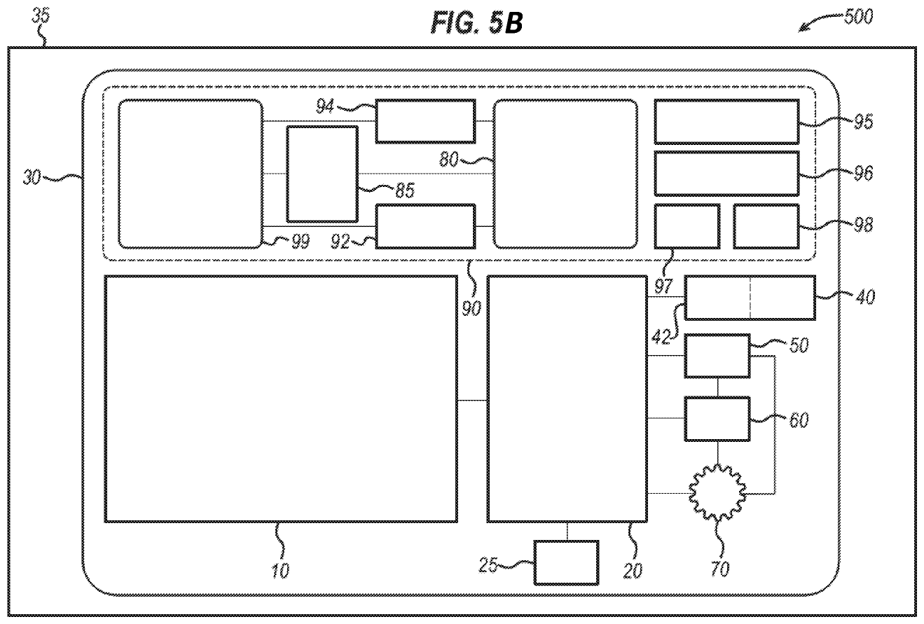

FIG. 5A is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 5A, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 70. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the temperature of the environment, location, and time. There may be a dedicated location sensor 98 which is configured to determine the geographical location of the device 500, e.g., via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatoirepical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

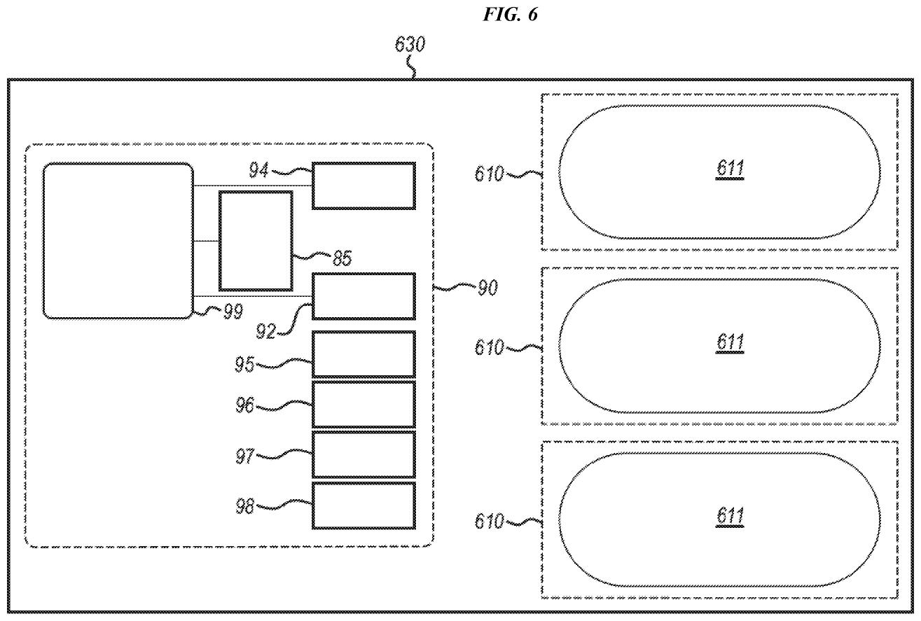

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 97, as described above.

### Electronic Communication

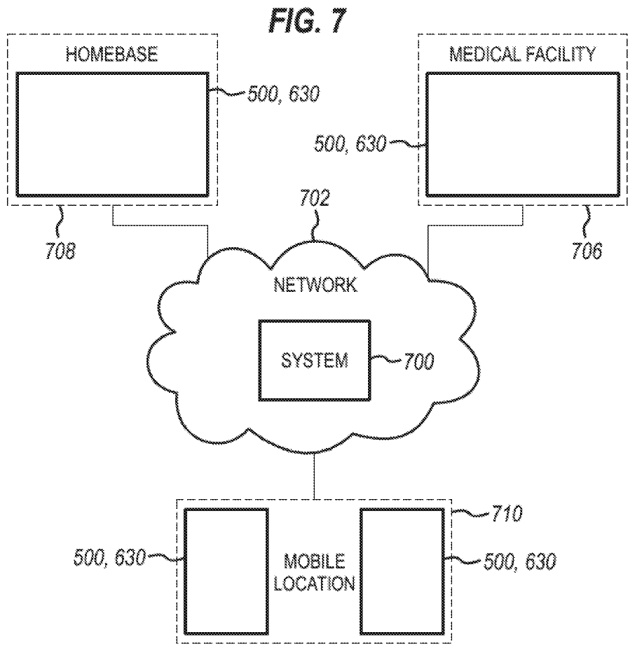

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

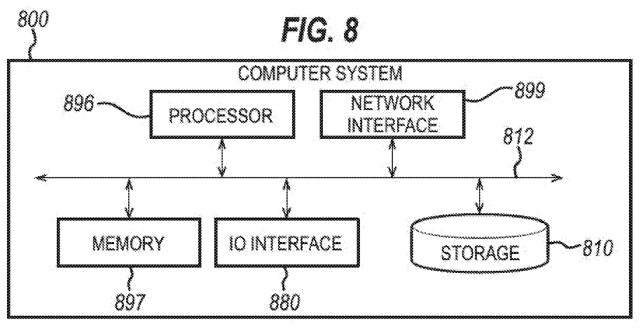

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (IO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For example, the memory 897 and storage device 810 can be integrated together or the communications interface 899 can be omitted if communication with another computer system is not necessary.

### Implementations

When delivering drugs using any of the drug delivery devices discussed above or any other drug delivery device, a variety of factors can influence drug administration, absorption, and effect on a patient beyond simply the initial drug dose itself. For example, individual patient physiologies or statuses, physiological effects on a patient of drug administration, surrounding external conditions to the patient, etc. can all influence results of drug administration on a patient. It can thus be beneficial to a patient to allow drug delivery to be adjusted based on a variety of different factors that arise during use of drug administration devices, providing more personalized treatment while also helping a patient receive and/or a doctor deliver specialized care for the specific patient being treated. Additionally, being able to automate as much of the drug delivery adjustment can help ease the process of delivery for both the patient and the doctor while improving patient outcomes.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

In at least some implementations, drug delivery can be altered based on one or more characteristics associated with the patient that are determined based on situational awareness of the patient. In an exemplary embodiment, a drug administration device includes at least first and second sensors that are each configured to gather data regarding a different characteristic associated with the patient. An algorithm stored on the device, e.g., in a memory thereof, is executable on board the device, e.g., by a processor thereof, to administer a dose of a drug to a patient. The algorithm is stored in the form of one or more sets of pluralities of data points defining and/or representing instructions, notifications, signals, etc. to control functions of the device and administration of the drug. Data gathered by the first and second sensors is used, e.g., by the processor, to change at least one variable parameter of the algorithm. The at least one variable parameter is among the algorithm's data points, e.g., are included in instructions for drug delivery, and are thus each able to be changed by changing one or more of the stored pluralities of data points of the algorithm. After the at least one variable parameter has been changed, subsequent execution of the algorithm administers another dose of the drug according to the changed algorithm. As such, drug delivery over time can be managed for a patient to increase the beneficial results of the drug by taking into consideration actual situations of the patient and actual results of the patient receiving doses of the drug. Changing the at least one variable parameter and/or administration of the one or more doses themselves is automated to improve patient outcomes. Thus, the drug administration device can be configured to provide personalized medicine based on the patient and the patient's surrounding conditions to provide a smart system for drug delivery.

Using the universal drug administration device 500 of FIG. 5B by way of example, the memory 97 can have stored therein the algorithm, and the processor 96 can be configured to execute the algorithm to control delivery of a dose of the drug dispensed by the dispensing mechanism 20. The processor 96 can also be configured to use data gathered by at least two of the one or more device sensors 92, environment sensor 94, and location sensor 98 to change at least one variable parameter of the algorithm such that a dose delivered subsequent to the changing of the at least one variable parameter will be controlled by execution of the changed algorithm. As mentioned above, a person skilled in the art will appreciate that the universal drug administration device 500 can be provided with a variety of the optional features described above, in a number of different combinations, e.g., not include any of the sensors 92, 94, 98 not being used by the processor 96 to change variable parameter(s) of the algorithm (and instead include sensor(s) to gather the needed situational awareness data), not include packaging 35, not include user interface 80, etc.

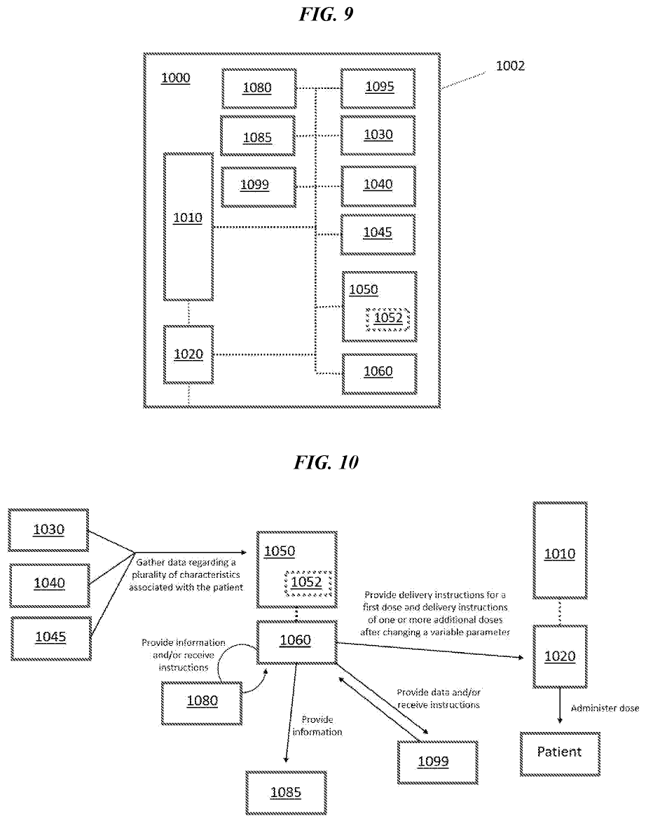

FIG. 9 illustrates one embodiment of a universal drug administration device 1000 configured to alter drug delivery to a patient based on one or more various characteristics associated with the patient that are determined based on situational awareness of the patient. The drug administration device 1000 in this illustrated embodiment includes a housing 1002, a drug holder 1010, a dispensing mechanism 1020, sensors 1030, 1040, 1045, a memory 1050 storing an algorithm 1052 therein that includes at least one variable parameter, a processor 1060, a user interface 1080, an indicator 1085, a power supply 1095, and a communications interface 1099. The sensors 1030, 1040, 1045 can each be configured to measure a different parameter, as discussed further below. Additionally, similar to that mentioned above regarding the universal drug administration device 500 of FIG. 5B, a person skilled in the art will appreciate that the universal drug administration device 1000 of FIG. 9 comprising the drug holder 1010, dispensing mechanism 1020, processor 1060, memory 1050, and sensors 1030, 1040, 1045 can be provided with a variety of the features described above, in a number of different combinations. For example, the device 1000 may include at least two sensors but not all of the sensors 1030, 1040, 1045, may not have a user interface 1080, etc.

First, second, and third sensors 1030, 1040, 1045 are each housed either within the housing 1002 or on an exterior surface of the housing 1002, and each sensor 1030, 1040, 1045 is configured to gather data regarding a characteristic associated with the patient. The sensors 1030, 1040, 1045 can each include a device sensor (similar to device sensor 92 discussed above), an environment sensor (similar to environment sensor 94 discussed above), or a location sensor (similar to location sensor 98 discussed above). Each of the sensors 1030, 1040, 1045 is configured to gathers data for a different characteristic. The characteristics can be physiological characteristics and/or situational characteristics of the patient. Various different physiological characteristics can be monitored, such as blood sugar level (e.g., using a glucose monitor, etc.), blood pressure (e.g., using a blood pressure monitor, etc.), perspiration level (e.g., using a fluid sensor, etc.), heart rate (e.g., using a heart rate monitor, etc.), respiratory rate (e.g., using a respiratory monitor, a heat sensor configured to be located near a nose or mouth and to use heat detection on the out-breath or detect in/out airflow movement, a pressure sensor configured to be located near a nose or mouth and to use pressure detection on the out-breath or detect in/out airflow movement, a spirometer, etc.), etc. Furthermore, a number of different situational characteristics can be monitored, such as core temperature, (e.g., using a temperature sensor), tremor detection (using an accelerometer, etc.), fall detection (using an accelerometer, etc.), irregular gait detection (using an accelerometer, etc.), time of day (e.g., using a timer, etc.), date (e.g., using a timer, etc.), patient activity level (e.g., using a motion sensor, etc.), blood pressure (e.g., using a blood pressure monitor, etc.), metabolic rate (e.g., using heart rate as discussed herein, etc.), altitude (e.g., using an altimeter, etc.), temperature of the drug (e.g., using a temperature sensor), viscosity of the drug (e.g., using a viscometer, using a viscosity versus temperature profile of the drug, etc.), GPS information (e.g., using a location sensor, etc.), weather information (e.g., using a temperature sensor, humidity sensor, etc.), room or external temperature (e.g., using a temperature sensor), angular rate (e.g., using an inertial measurement unit (IMU) or MARG (magnetic, angular rate, and gravity) sensor), body orientation (e.g., using an IMU, etc.), current of a motor used in delivering the drug (e.g., using a current sensor), blood oxygenation level (e.g., using a blood oxygen sensor), sun exposure (e.g., using a UV sensor, etc.), osmolality (e.g., using a blood monitor, etc.), and air quality (e.g., using a UV sensor, etc.), inflammatory response, one or more images and/or videos of the patient and/or an environment in which the patient is located (for example, to analyze food intake; to determine whether solid food or liquid is being consumed; to determine a location or activity of the patient; to determine a condition of the patient such as skin reaction, breathing, eye dilation, sedation, disassociation, voice characteristics such as tone and pitch; etc.), user-input data such as general well-being, pain score, or a cycle time between flare ups of a particular ailment, etc. In an exemplary embodiment, one sensor 1030, 1040, 1045 is configured to monitor either a physiological or a situational characteristic and the others of the sensors 1030, 1040, 1045 are configured to monitor the other of physiological or situational characteristics. In another exemplary embodiment, each of the sensors 1030, 1040, 1045 is configured to monitor a different physiological characteristic. In another exemplary embodiment, each of the sensors 1030, 1040, 1045 is configured to monitor a different situational characteristic.

While three sensors are illustrated in FIG. 9, the device 1000 can include only two sensors or can include more than three sensors. Any additional sensors can be configured similarly to sensors 1030, 1040, 1045 and be configured to monitor different characteristics than the sensors 1030, 1040, 1045 and from each other.

The memory 1050 of the device 1000 is located in the housing 1002. In this illustrated embodiment, the memory 1050 is configured to store data from the sensors 1030, 1040, 1045, however in other embodiments this data can be stored elsewhere, such as in another memory on board the device 1000 and/or in a remote memory accessible to the device 1000 via the communications interface 1099. The algorithm 1052 stored in the memory 1050 represents instructions for the device 1000 regarding how to administer the drug in the drug holder 1010 and is configured to be executed by the processor 1060. The algorithm 1052 is stored in the form of a plurality of data points defining and/or representing instructions, notifications, signals, etc. to control drug administration, with the at least one variable parameter being among the data points such that changing the at least one variable parameter of the algorithm 1052 results in at least one change in how the drug is administered. The at least one variable parameter can be any of a variety of different delivery and/or drug parameters. Examples of variable parameters include a rate of delivery of the drug from the drug holder 1010 to the patient, a time interval between dose deliveries such that doses delivered after the at least one variable parameter is changed are at a different time interval than doses delivered before the change, a dosage amount, a dosage concentration, whether or not any additional doses are delivered such as stopping a second or any subsequent dose or starting to dose again after dosing was previously stopped before a first dose or before any subsequent dose after the first dose, etc.

The processor 1060 is configured to receive and analyze data from the one or more sensors 1030, 1040, 1045 and to execute the algorithm 1052 to control administration of one or more doses of the drug to the patient. In an exemplary embodiment, the processor 1060 executes the algorithm 1052 to control delivery of at least a first dose of the drug to the patient, changes the at least one variable parameter of the algorithm 1052 based on data gathered by the sensors 1030, 1040, 1045, and executes the algorithm 1052 after changing the at least one variable parameter to control delivery of at least one subsequent dose of the drug. In some embodiments, the processor can change the at least one variable parameter of the algorithm 1052 based on data gathered by the sensors 1030, 1040, 1045 before execution of the algorithm 1052 to control delivery of the first dose, such as by changing a variable parameter from indicating that dosing was stopped (e.g., because the drug administration device's device operation prevention mechanism is in a state to prevent drug delivery, the power supply 1095 lacks sufficient power to deliver a dose, etc.) to indicating that dosing is allowed (e.g., the drug administration device's device operation prevention mechanism is in a state to allow drug delivery, the power supply 1095 has sufficient power to deliver a dose, etc.). To execute the algorithm 1052, the processor 1060 is configured to run a program stored in the memory 1050 to access the plurality of data points of the algorithm 1052 in the memory 1050. To change the at least one variable parameter of the algorithm 1052, the processor 1060 is configured to modify or update the data point(s) of the at least one variable parameter in the memory 1050. The processor 1060 can also be configured to execute instructions stored in the memory 1050 to control the device 1000 generally, including other electrical components thereof such as the communications interface 1099, indicator 1085, and user interface 1080. The processor 1060 can be configured to change the at least one variable parameter of the algorithm 1052 during the delivery of a dose such that the algorithm 1052 is changed in real time with the delivery of the dose, which may accommodate real time sensed conditions, or the processor 1060 can be configured to change the at least one variable parameter of the algorithm 1052 before a start of the delivery of the dose, which may consume less memory and use fewer processing resources during algorithm 1052 execution than real time changing of the algorithm 1052.

The processor 1060 can be configured to automatically control delivery of doses of the drug based on one or more predetermined schedules or intervals of dosing for the patient, which can be predetermined prior to an initial dose or can be determined during use of the device 1000 after delivery of the first dose and set such that future doses can be based on the predetermined schedule(s). The predetermined schedule(s) can also be determined by a doctor or other care provider, created automatically based on the algorithm 1052 and/or the sensors 1030, 1040, 1045 being used, or some combination of the two.

The processor 1060 can be configured to cause a notification to be provided to the patient and/or the doctor or other care provider based on gathered data from one or more of the sensors 1030, 1040, 1045, for example via the device indicator 1085, user interface 1080, and/or communications interface 1099.

Because the processor 1060 is configured to alter the at least one variable parameter based on data gathered by one or more of the sensors 1030, 1040, 1045, an automated reaction response based on the situational awareness of the patient is possible. In at least some embodiments, the at least one variable parameter is altered to provide adaptive dose adjustment based on various readings and/or data from one or more of the sensors 1030, 1040, 1045 and/or user inputs. For example, a user can record a cycle time between flare ups of a disease or ailment, at which point the drug dosing schedule as reflected in the algorithm 1052 can be adjusted by the processor 1060 and/or remotely by a doctor or other care provider to take this into account for better disease control. As another example, changes in altitude of a patient can potentially alter the effectiveness of medications and even lead to toxicity in some cases. As such, the duration at which a patient is at a different altitude can be read by one or more of the sensors 1030, 1040, 1045 and be used by the processor 1060 and/or a doctor or other care provider to adjust subsequent drug dosages by the processor 1060 changing the at least one variable parameter. In another example, a treatment can be discontinued entirely based on one or more sensor readings, and a patient can be informed as such through the device indicator 1085 and/or user interface 1080. One or more possible complications can be anticipated based on best practices, and the processor 1060 and the memory 1050 can operate together to provide various digital ready reactions to common complications (identified through situational awareness) to alert the patient, attempt to change a behavior of the patient, notify the doctor, etc. In various embodiments, at least one of the sensors 1030, 1040, 1045 includes a camera, and the processor 1060 is configured to analyze image(s) and/or video(s) captured by the camera, such as to analyze any food intake and/or determine one or more side effects such as patient skin reaction to the drug, patient sedation level, patient disassociation level, vomiting, etc. Facial ID can be used to identify the patient in image(s) captures by the at least one of the sensors 1030, 1040, 1045 including a camera to help ensure that relevant data is being gathered and analyzed.

The device indicator 1085 and/or the user interface 1080 can be configured to operate independently of each other or configured to operate together to provide various notifications to the patient and/or a doctor or other care provider of any outputs of situational awareness based on sensor readings and any complications those readings could indicate. As such, a patient may be able to react quickly to any negative results of administration of the drug and/or complications as a result of treatment. In at least some embodiments the device 1000 can be configured to provide activities the patient can pursue to best manage their condition, such as by providing suggested activities via the user interface 1080. Furthermore, information of situational awareness based on sensor readings and any complications those readings could indicate can be relayed to the patient's doctor or other care provider, who can then communicate with the patient.

Basic operations on the device 1000 itself can inform the patient of any detected deviation from the so-called “five rights” of drug administration in at least some embodiments. During drug administration, best practices require ensuring that the “five rights” of medication use are followed: the right patient, the right drug, the right time, the right dose, and the right route. Tracking situational awareness of the patient through use of the sensors 1030, 1040, 1045 can thus detect if any of the “five rights” are violated, at which point the patient and/or a doctor or other care provider can be informed. Furthermore, in some embodiments, confirmation of the “five rights” can be required, either by a doctor or off-site medical personnel or by the patient themselves, to help eliminate medication errors and/or to ensure that the drug's Risk Evaluation and Mitigation Strategies (REMS) is followed. In severe cases, a notification can be provided to the patient and/or the doctor or other care provider that immediate medical attention is needed.

The communications interface 1099 can be configured to allow one-way communication, such as providing data to a remote server and/or receiving instructions or commands from a remote server, or two-way communication, such as providing information, messages, data, etc. regarding the device 1000 and/or data stored thereon and receiving instructions, such as from a doctor, a remote server regarding updates to software, etc. As such, doctor/care provider interaction is possible to provide additional adjustments to care. For example, doctors or other care providers can receive relevant information and data from the device 1000 via the communications interface 1099 and/or from the patient directly and can, based on the received information, provide remote feedback and/or any adjustment(s) to the device 1000 (for example, requesting that the processor 1060 change the at least one variable parameter to change subsequent dosing) and/or to the patient (for example, providing recommended next steps based on current sensor readings and feedback from the patient). In at least some embodiments any recording or data of an incident and the data leading up to and resulting from the incident can be provided to the doctor and/or remote-care individuals and/or a remote server for storage, and the receiving party can analyze and summarize the data to determine a recommendation regarding overcoming or effectively addressing any current complication. In at least some embodiments, the processor 1060 is configured to change the at least one variable parameter only after communicating with a remote server and receiving any instructions therefrom.

While the sensors 1030, 1040, 1045 are all included with the device 1000 in this illustrated embodiment, one or more of the sensors 1030, 1040, 1045 can be separate from the device 1000 by, e.g., being worn on the patient, being placed in a shared geographical space with the patient, being attached to other equipment or instruments, being part of a mobile phone app used by the patient, etc.

Changing the at least one variable parameter can result in an adjusted injection or flow rate speed of each provided dose, e.g., the at least one variable parameter can include injection or flow rate speed. Instead or in addition, a temperature of a drug can be varied to create constant flow, as discussed in U.S. Patent Pub. No. 2002/0042596 entitled “Method And Apparatus To Sense Temperature In An Implantable Pump” published Apr. 11, 2002 and hereby incorporated by reference in its entirety.

Drug delivery site pain can be minimized in at least some embodiments by the device 1000 monitoring patient usage, patient preferences, patient physical attributes, the one or more sensed characteristics associated with the patient, various parameters of the drug, etc. For example, patient factors such as weight, BMI, age, etc.; type of delivery mechanism of the drug, such as bolus injection delivery, continuous delivery, inhalation, nasal spray, etc.; a total number and history of injections at the desired location for injectable drugs; a volume of the drug; measured parameters about a status and state of the drug itself like viscosity, temperature, pH level; can all be used to anticipate the pain involved in administering the next drug administration, such as the next injection, next inhalation, next nasal spray, etc. Various delivery parameters of the drug, such as the speed, wait period, pressure, location recommendations, etc. can then be updated through the at least one variable parameter to minimize patient pain and discomfort.

In at least some embodiments, allowing the device 1000 to have more situational awareness of the patient may facilitate patient compliance. The drug administration device 1000 can be used to increase compliance by a patient and/or increase familiarity with how the device 1000 operates in any number of different ways. For example, the device 1000 can be configured to provide reminders, updates, adaptive training, etc. to the patient based on patient compliance data and/or patient compliance data that is stored on or pushed out to the device 1000 to reinforce healthy behaviors, teach steps of use for the device, and other details. Increasing compliance and familiarity with the device 1000 can help reduce patient risk when being administered the drug, the importance of which is discussed, for example, in U.S. Patent Pub. No. 2015/0359966 entitled “System For Monitoring And Delivering Medication To A Patient And Method Of Using The Same To Minimize The Risks Associated With Automated Therapy” published Dec. 17, 2015 and hereby incorporated by reference in its entirety.

FIG. 10 illustrates an embodiment of use of the drug administration device 1000. Prior to a first delivery of a dose of the drug, the drug administration device 1000 gathers data regarding the first characteristic associated with the patient using the first sensor 1030, gathers data regarding the second characteristic using the second sensor 1040, and gathers data regarding the third characteristic using the third sensor 1045. The first delivery of the dose can be the initial dose delivered from the device 1000 to the patient, or it can be the first dose delivered from the device 1000 to the patient after at least one dose has already been provided to the patient and after a sufficient amount of data has been gathered via the sensors 1030, 1040, 1045. Further data can optionally be gathered regarding additional characteristics associated with the patient by using additional secondary sensors. The data gathered represents pluralities of data points defining each characteristic and are stored in the memory 1050. The processor 1060 subsequently controls delivery of the first dose of the drug from the device 1000 to the patient by executing the algorithm 1052 stored in the memory 1050. The sensors 1030, 1040, 1045 continue gathering data. Based on any of this subsequently gathered data, the processor 1060 changes the at least one variable parameter of the algorithm 1052. After changing the at least one variable parameter, the processor 1060 controls delivery of at least the second dose from the device 1000 to the patient by executing the algorithm 1052. The processor 1060 executing the algorithm 1052 to deliver a dose can be automatic, manual, or some combination of the two, and it can be according to a predetermined schedule of dosing, as discussed above.

During any part of the dosage process of FIG. 10, the device 1000 can communicate with one or more remote computer systems using the communications interface 1099 to provide data thereto and/or receive instructions therefrom and/or can communicate with the user via the device indicator 1085 and/or the user interface 1080 to provide information thereto and/or receive instructions therefrom. In some embodiments, administering a first dose, a second dose, and/or any subsequent doses can be dependent on receiving instructions from one or more remote computer systems. Furthermore, the second or any subsequent doses can also be prevented entirely based on changes to the variable parameter, thus resulting effectively in the second or subsequent dose being equivalent to zero drug being administered.

It may be desirable to prevent the second or any subsequent doses, or even the first dose, from being administered from the drug administration device 1000 for any of a variety of reasons. For example, one of the sensors 1030, 1040, 1045 can be configured to monitor the patient's location, e.g., GPS or other location information. The drug administration device 1000 may be expected to be used only at a certain location, such as at a hospital or other medical care facility where the patient must receive the drug from the drug administration device 1000 because, e.g., the drug is a controlled substance such as esketamine or ketamine that must be administered in a controlled facility, the patient is still learning how to correctly use the drug administration device 1000 and is in a period of observation for use of the device 1000, etc. The drug administration device 1000 may be expected to be used only in any of a plurality of hospitals or other medical facilities certified to provide the drug to patients such as when the drug is esketamine, ketamine, or other controlled substance, in a particular city where the patient resides, in a particular city where the patient has pre-registered as visiting on a certain day/time, etc. The patient may be expected to stay at a location of drug administration to help ensure that any side effects of the drug delivered from the drug administration device 1000 dissipate before the patient drives or otherwise leaves the location of drug administration (e.g., is driven by another person, walks, etc.) such that the patient's location changing before a predetermined threshold amount of time has elapsed since delivery of a drug dose can disqualify the patient from being able to receive the drug from the drug administration device 1000 in the future. If the one of the sensors 1030, 1040, 1045 monitors the patient's location to not be at the expected location for drug administration and/or for a predetermined threshold amount of time has elapsed since delivery of a drug dose, the at least one variable parameter can be changed (or maintained) to effectively make the subsequent dose equivalent to zero drug being administered. The drug administration device 1000 may be expected to be used by a particular patient only in a particular one or more locations (e.g., at a particular GPS location, at the patient's primary doctor's office, at the patient's primary hospital, at the patient's home, etc.), such as when the drug is esketamine, ketamine, or other controlled substance and location of the drug's use may be important in helping to ensure that the drug has not been diverted or is being used by an unauthorized party. For some drugs, such as esketamine, ketamine, or other controlled substances, the drug's REMS may require that the location of drug administration is recorded. The patient may be expected to stay at the location of drug administration to help ensure that any side effects of the drug delivered from the drug administration device 1000 dissipate before the patient drives or otherwise leaves the location of drug administration (e.g., is driven by another person, walks, etc.) such that the patient's location changing before a predetermined threshold amount of time has elapsed since delivery of a drug dose can disqualify the patient from being able to receive the drug from the drug administration device 1000 in the future. If the one of the sensors 1030, 1040, 1045 monitors the patient's location to not be at the expected location for drug administration and/or for a predetermined threshold amount of time has elapsed since delivery of a drug dose, the at least one variable parameter can be changed (or maintained) to effectively make the subsequent dose equivalent to zero drug being administered. In some instances, the particular patient's expected location of drug administration is the patient's home. If the one of the sensors 1030, 1040, 1045 monitors the patient's location to not be at the expected location for drug administration and/or for a predetermined threshold amount of time has elapsed since delivery of a drug dose, the patient may be disqualified from being able to administer the drug at home and instead be required to be at a doctor's office or other medical care facility for drug administration or the drug administration device will switch to a locked state in which drug delivery is prevented from the drug administration device.

For another example, one of the sensors 1030, 1040, 1045 can be configured to monitor an angular orientation of the drug administration device 1000, e.g., using an accelerometer, a gyro, a tilt/angle switch (mercury free), a position sensor, etc. Some drug administration devices should be at a particular angular orientation relative to the patient during drug administration to help ensure that the drug is delivered properly. For example, a proper angular orientation of an injection device can be a vertical, substantially perpendicular orientation, e.g., substantially 90°, relative to the patient's skin versus an improper position of being at a non-perpendicular angle relative to the patient's skin. A person skilled in the art will appreciate that the angle may not be precisely perpendicular (precisely 90°) but nevertheless be considered to be substantially perpendicular for any of a variety of reasons, such as manufacturing tolerance and sensitivity of measurement equipment. For another example, a proper angular orientation of a nasal spray device can be in a range of 30° to 60°, in a range of 30° to 40°, in a range of 30° to 50°, in a range of 40° to 50°, in a range of 50° to 60°, or in a range of 40° to 60°. The one of the sensors 1030, 1040, 1045 being configured to monitor an angular orientation of the drug administration device 1000 can allow for detecting an angular orientation of the drug administration device 1000 to allow for determining whether the drug administration device 1000 is in a proper angular orientation for drug delivery. If the one of the sensors 1030, 1040, 1045 monitors the patient's location to not be at the proper angular orientation for drug delivery, the at least one variable parameter can be changed (or maintained) to effectively make the subsequent dose equivalent to zero drug being administered. When the proper angular orientation is detected, the at least one variable parameter can be changed from zero to allow the drug to be administered.

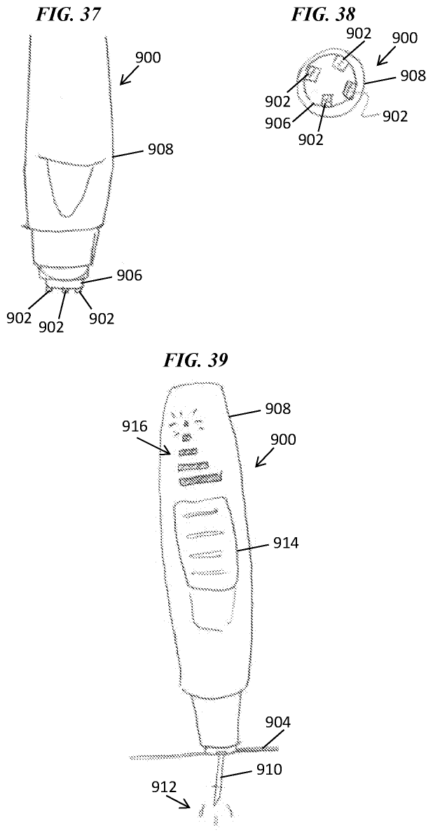

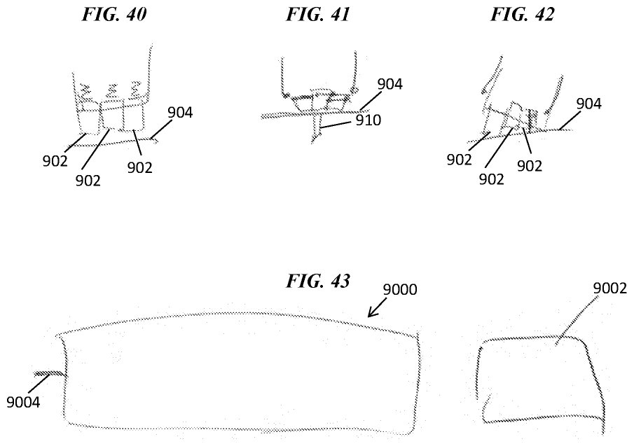

FIGS. 37-39 illustrate one embodiment of a drug administration device 900 that should be at a particular angular orientation relative to a patient during drug administration to help ensure that drug is delivered properly to the patient from the drug administration device 900. The drug administration device 900 in this illustrated embodiment is an autoinjector, e.g., the autoinjector 100 of FIG. 1. The proper angular orientation of the autoinjector 900 for drug delivery is a vertical, substantially perpendicular orientation relative to a patient's skin, while an improper position of the autoinjector 900 for drug delivery is at a non-perpendicular angle relative to the patient's skin. FIG. 40 illustrates the autoinjector 900 relative to a patient's skin 904 before the autoinjector 900 contacts the skin 904. FIGS. 39 and 41 illustrate the autoinjector 900 at the proper angular orientation of the autoinjector 900 relative to the skin 904 after the autoinjector 900 has contacted the skin 904 and the autoinjector's dispensing mechanism protection mechanism, in the form of a needle shield 906 in this illustrated embodiment, has been pushed into a housing 908 of the autoinjector 900 and a needle 910 of the autoinjector 900, previously shielded by the needle shield 906, has penetrated the skin 904 in response to the autoinjector 900 contacting and being pushed toward the skin 904. FIG. 39 illustrates the autoinjector 900 before drug delivery. FIG. 41 illustrates the autoinjector 900 during drug delivery with drug 912 exiting the needle 910 into the patient. FIG. 42 illustrates the autoinjector 900 at one of a plurality of possible improper angular orientations of the autoinjector 900 relative to the skin 904 after the autoinjector 900 has contacted the skin 904 and the needle shield 906 has been pushed partially into the housing 908 in response to the autoinjector 900 contacting and being pushed toward the skin 904. The needle shield 906 has only partially advanced into the housing 908 due to the improper angular orientation.

The autoinjector 900 includes at least one sensor 902 configured to monitor an angular orientation of the drug administration device 900. The at least one sensor 902 extends distally from the needle shield 906. The at least one sensor 902 is operatively coupled to the needle shield 906 such that movement of the needle shield 906, e.g., sliding of the needle shield 906 in a proximal direction into the housing 908, also causes movement of the at least one sensor 902. The sensors 902 are arranged equidistantly around a perimeter of the needle shield 906, as shown in FIG. 38, which allows for data to be gathered from different areas and for a more confident assessment to be made of the autoinjector's angular orientation relative to the skin 904. In an exemplary embodiment, the at least one sensor 902 includes a plurality of sensors, as in this illustrated embodiment, to help allow for data to be gathered from different areas and for a more confident assessment to be made of the autoinjector's angular orientation relative to the skin 904. The at least one sensor 902 includes four sensors in this illustrated embodiment, but as discussed herein, another number of sensors can be used. One of the sensors 902 is obscured from view in each of FIGS. 37, 40, and 42.

Each of the sensors 902 in this illustrated embodiment is a contact sensor configured to measure contact thereof with a surface. If all of the sensors 902 are determined, e.g., by a processor of the autoinjector 900, to be in direct contact with a surface, e.g., a surface of the skin 904, the autoinjector 900 can be considered to be in the proper angular orientation for drug delivery. If any one or more of the sensors 902 are determined, e.g., by the processor of the autoinjector 900, to not be in direct contact with a surface, e.g., the surface of the skin 904, the autoinjector 900 can be considered to be in the improper angular orientation for drug delivery.

In another embodiment, each of the sensors 902 can be pressure sensor configured to measure pressure. If all of the sensors 902 are determined, e.g., by the processor of the autoinjector 900, to be measuring substantially the same pressure, the autoinjector 900 can be considered to be in the proper angular orientation for drug delivery. The sensors 902 all measuring the same pressure is indicative that all of the sensors 902 have been pressed equally against the skin 904 for level contact of the autoinjector 900 against the skin 904 such that the autoinjector 900 is substantially perpendicular to the skin 904. If any one or more of the sensors 902 are determined, e.g., by the processor of the autoinjector 900, to not be measuring substantially the same pressure as the other sensor(s) 902, the autoinjector 900 can be considered to be in the improper angular orientation for drug delivery. The sensors 902 not all measuring the same pressure is indicative that not all of the sensors 902 have been pressed equally against the skin 904 for level contact of the autoinjector 900 against the skin 904 such that the autoinjector 900 is not substantially perpendicular to the skin 904.

In response to the autoinjector 900 having been determined to be in the proper angular orientation based on data gathered by the at least one sensor 902, the autoinjector 900, e.g., the processor thereof, can be configured to cause the autoinjector 900 to move from a locked state, in which drug delivery is prevented, to an unlocked state, in which drug delivery is allowed. In the locked state, the autoinjector's trigger 914 cannot be pressed to inject the drug 912 into the patient. In the unlocked state, the trigger 914 can be pressed to inject the drug 912 into the patient. The autoinjector 900 can be moved from the locked state to the unlocked state in a variety of ways. For example, the autoinjector 900, e.g., the processor thereof, can change a variable parameter of an algorithm that controls dose delivery as discussed herein, to effectively make the subsequent dose equivalent to zero drug being administered. For another example, the autoinjector 900 can include a device operation prevention mechanism that the autoinjector 900, e.g., the processor thereof, causes to move from its locked state to its unlocked state in response to the autoinjector 900 having been determined to be in the proper angular orientation based on data gathered by the at least one sensor 902.

The autoinjector 900 includes a user interface 916 configured to provide information to a user of the autoinjector 900, as described herein. The light allows a user of the autoinjector 900 to easily see whether or not the autoinjector 900 is in a correct position for drug administration. The user interface 916 in this illustrated embodiment includes a light configured to be illuminated when the autoinjector 900 is in the proper angular orientation and to not be illuminated when the autoinjector 900 is in the improper angular orientation. In other embodiments, the light can be configured to illuminated in a first color when the autoinjector 900 is in the proper angular orientation and to be illuminated in a second, different color when the autoinjector 900 is in the improper angular orientation. The autoinjector's processor is configured to control the light's illumination. The user interface 916 can have other configurations, as described herein.

The light in this illustrated embodiment includes a plurality of light strips of increasingly shorter length in a proximal direction. The light includes five light strips in this illustrated embodiment, but another number of light strips can be used. Additionally, a different style of light can be used. The autoinjector 900, e.g., the processor thereof, is configured to sequentially illuminate the light strips in a proximal direction after the trigger 914 is pressed to visually signal to the user a countdown to drug delivery, e.g., ejection of the drug through the needle 910. FIG. 39 shows all of the light strips illuminated, thereby indicating that drug delivery is occurring. Informing a user when drug delivery will begin and when drug delivery is occurring may help provide user confidence that the autoinjector 900 is working properly.

For yet another example regarding prevention of the second or any subsequent doses, or even the first dose, from being administered from the drug administration device 1000, one of the sensors 1030, 1040, 1045 can be configured to monitor elapsed time, e.g., using a time, a counter, etc. Some drug administration devices should not deliver a second dose until after a certain amount of time has passed since delivery of a first dose. For example, a certain amount of time elapsing between doses delivered by a nasal spray device that delivers a spray into one nostril at a time may help ensure that the nasal spray device has been moved from one nostril to another before the second dose is delivered. For another example, a certain amount of time elapsing between doses may help prevent overdose. If the one of the sensors 1030, 1040, 1045 monitors elapsed time since the first dose to not be less than a predetermined threshold amount of time, the at least one variable parameter can be changed (or maintained) to effectively make the subsequent dose equivalent to zero drug being administered. When the predetermined threshold amount of time has passed, the at least one variable parameter can be changed from zero to allow the drug to be administered.

FIG. 43 illustrates another embodiment of a drug administration device 9000 configured to visually signal to the user a countdown to drug delivery from the drug administration device 9000. In this illustrated embodiment, a powered add-on module 9002 is configured to be attached to the drug administration device 9000 and to provide the countdown to drug delivery on a user interface of the add-on module 9002, e.g., on a display thereof, using light(s), using sound, etc. The add-on module 9002 includes an on-board power supply configured to provide power to the user interface of the add-on module 9002. The drug administration device 9000 is an autoinjector including a needle 9004 in this illustrated embodiment, but other types of drug administration devices can be used with the add-on module 9002.

The add-on module 9002 is configured to be attached to a proximal end of the drug administration device 9000 opposite to a distal end of the drug administration device 9000, e.g., the end of the drug administration device 9000 at which the needle 9004 is located. The add-on module 9002 can be configured to be non-removably attached to the drug administration device 9000 prior to a user receiving the drug administration device 9000, which may help ensure that the drug administration device 9000 is used with the add-on module 9002. Alternatively, as in this illustrated embodiment, the add-on module 9002 can be configured to be removably attached to the drug administration device 9000 by a user of the drug administration device 9002 or by another entity. The add-on module 9002 being removable may allow the add-on module 9002 to be used with each of a plurality of drug administration devices and thereby make the add-on module 9002 more cost efficient. The add-on module 9002 can be non-removably attachable to the drug administration device 9000 by, for example, a distal end of the add-on module 9002 including a cavity configured to securely seat a trigger button at the proximal end of the autoinjector 9000 therein, such as by press fit.

Whether removably or non-removably attached to the autoinjector 9000, the add-on module 9002 is configured to operatively connect to a trigger of the autoinjector 9000. In an exemplary embodiment, the trigger is a trigger button at the proximal end of the autoinjector 9000. The add-on module 9002, as in this illustrated embodiment, can have a larger proximal surface area than the underlying trigger button, which may make actuation of the drug administration device 9000 easier for at least some users, such as those with limited dexterity and/or strength. The add-on module 9002, attached to the autoinjector 9000, is configured to be pushed to actuate the trigger, e.g., to push the button, and cause drug delivery. The user interface of the add-on module 9002 is configured to provide a countdown to drug delivery from the drug administration device 9000 similar to that discussed above. A start of the countdown is in response to the pushing of the add-on module 9002 and the trigger button. The add-on module 9002 can include a switch configured to be open prior to the add-on module 9002 being pushed and to close in response to the add-on module 9002 being pushed. The switch closing can cause a circuit to close, thereby triggering the countdown to begin. The add-on module 9002 can include a processor configured to control the user interface. The user interface can also be configured to indicate that drug delivery is occurring, similar to that discussed above regarding the light strips, although as mentioned above the user interface can provide information in a way other than using light(s).

In at least some embodiments, the processor 1060 is configured to use a hierarchy in terms of how data from the sensors 1030, 1040, 1045 is used compared to each other and/or to any additional sensors. The hierarchy prioritizes one of the sensors over the other(s) such that one acts as a primary sensor, such as sensor 1030, and the other(s) act as secondary or ancillary sensor(s), such as sensors 1040, 1045. In such embodiments, the characteristic measured by the primary sensor can be considered to be the primary or defining characteristic, and characteristics measured by the secondary sensors can be secondary or influencing characteristics on the primary characteristic. This prioritization or hierarchy of characteristics (and thus data) can be helpful when the drug administration device 1000 is used for a treatment that includes one controlling characteristic and one or more secondary characteristics that may influence or assist in monitoring the controlling characteristic, for example when measuring blood pressure when administering blood pressure medication or when measuring blood sugar level when administering insulin. While secondary characteristics can help in monitoring high blood pressure or low blood sugar, the characteristics of primary concern in each example is blood pressure itself or blood sugar level itself, as discussed in detail below. The prioritization of data and inputs from one or more secondary sensors based on the hierarchical relationship can be customizable based on desired patient outcomes, various expected or anticipated side-effects, the drug being administered, time of day, location, activity level, caloric intake, physical activity, etc. The device 1000 can thus have a predefined hierarchy of levels or severity of effect on dosage based on the sensed characteristics from the sensor(s). A medical professional or unlearned algorithm within the device 1000 itself can optionally adjust the priority of the levels or reorder the importance of the various sensed data and inputs as a result of dosing amounts and/or dosing timelines, as discussed below. Because so much data can be generated by using a plurality of sensors and because data from one sensor may contradict data from another sensor in some instances, effectively using situational awareness to personalize drug administration to each patient may benefit from prioritization and relative weighing of multiple sources of information to arrive at a most correct conclusion or recommendation to best help the patient. This hierarchy of prioritization can be customized for a specific patient based on how the patient presents in any one moment or over time, therefore providing an adaptive device with re-orderable hierarchal relationships.

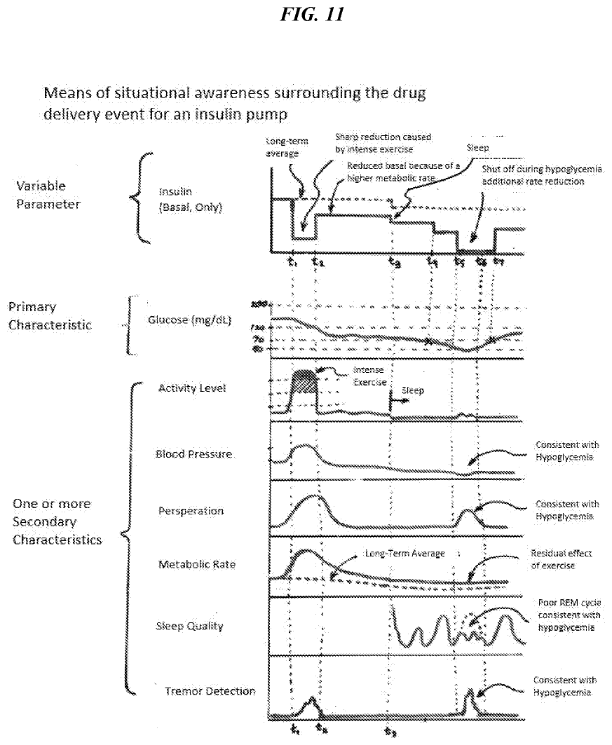

As mentioned above, the hierarchical arrangement can be used in a variety of ways, for example to verify a physiological result such that data from one or more sensors is considered to adjust the at least one variable parameter to proactively manage any anticipated negative effect on the primary characteristic being measured. As one example, FIG. 11 illustrates a chart tracking use of the drug administration device 1000 as an insulin pump, although as discussed above the drug administration device 1000 can be another type of device. The at least one variable parameter of the insulin pump includes insulin level being delivered to a patient over time by the pump. The first sensor 1030 is designated as the primary sensor and is configured to measure a primary characteristic in the form of a glucose level in the patient. The designated secondary sensors 1040, 1045 are each configured to track secondary characteristics or measurements, which in this example include activity level of the patient, blood pressure, perspiration, metabolic rate, sleep quality, and tremor detection, each of which may be sensed with a different sensor such that more than three sensors are used. Tracking glucose levels as the primary characteristic and varying delivered insulin levels allows the insulin pump, e.g., the processor 1060 thereof, to determine a typical long-term average or basal insulin level that the patient is expected to receive, which allows the insulin pump generally to modify how much insulin it expects to deliver to the patient over time. As illustrated, the average insulin level can stay consistent throughout the day, and then it can be decreased during the night when the patient is usually sleeping. Thus, tracking the primary characteristic allows some modifications to the insulin levels being delivered. However, it is not very personalized to the patient. When one or more secondary characteristics are tracked, more personalized and specialized care is possible because the insulin pump can proactively manage dosing or administration of insulin. As such, the pump can assist in maintaining a healthy glucose level (for example, between about 50 and 200 mg/dL and more preferably between about 70 and 120 mg/dL) in the patient on an ongoing basis instead of waiting to detect various negative outcomes of poorly managed insulin and glucose levels, such as hypoglycemia triggered by a very low glucose level (for example, about 50 mg/dL or less), to then correct dosage levels.

Initially, the patient receives a baseline amount of insulin from the insulin pump via execution of the algorithm 1052, and glucose levels in the patient as determined by the glucose (primary) sensor are within a healthy range. At time t₁, however, the insulin pump detects the onset of intense exercise through one or more of the secondary sensors, e.g., activity level sensor can detect intense activity, blood pressure sensor detects an increase in blood pressure of the patient, perspiration sensor detects an increased amount of sweating, metabolic rate sensor increases significantly, and tremor detection sensor can detect possible tremors in the patient. All of these sensor readings can be analyzed together by the processor 1060 to allow the insulin pump to determine that the patient is most likely exercising based on predefined criteria categorizing sensor data from the various sensors as being in a range or having a value that is indicative of exercise. The insulin pump can then lower dosing amounts and/or dosing intervals, by changing the at least one variable, to reduce the amount of insulin being provided to the patient to compensate for exercising and thus maintain a healthy glucose level.

At time t₂, the insulin pump determines that exercising has most likely terminated according to predefined criteria, e.g., because the activity level sensor and the tremor detection sensor both stop detecting movement and the other sensors show gradual returns to a normal or resting rate. Thus, the insulin pump increases dosing amounts and/or dosing intervals, e.g., changes the same at least one variable previously changed, to compensate for the termination of exercising. However, the levels are not returned to pre-dosing levels because of the residual effects of exercising on the patient, for example an increased metabolic rate that will remain elevated above a long-term average for at least several hours after exercising.

At time t₃, the insulin pump reduces dosing amounts and/or dosing intervals because the pump determines, based on predefined criteria, that the patient has most likely gone to sleep based on a complete lack of detected readings from the activity level sensor. Because of the hierarchical nature of the sensors, readings from the sleep quality sensor can be prioritized last or not taken at all during active hours when the patient is not sleeping. When the patient begins to sleep, however, the priority of the sleep quality sensor can be increased to more prominently influence behavior of the insulin pump. Alternatively, the sleep quality sensor can be manually activated by the patient or can be automatically activated based on various readings from the secondary sensors only during sleeping hours.

At time t₅, insulin delivery can be terminated entirely by the insulin pump because of a possible hypoglycemic event being detected. Glucose levels can lower significantly, activity level can begin to increase, blood pressure can dip, perspiration can increase dramatically, a poor REM cycle of sleep can be detected by the sleep quality sensor, and possible tremors can be detected by the tremor detection sensor. These measured characteristics can be analyzed by the insulin pump, which can identify each indicator as being consistent with hypoglycemia, and the pump thus terminates insulin delivery by changing the at least one variable parameter to reflect no doses, e.g., by changing dose amount to zero or by changing dose frequency to a never-achievable time period. Because dosing is terminated promptly at the beginning of the possible hypoglycemic event rather than waiting until glucose level falls below a normal or safe threshold and only then reacting, the glucose level in the patient begins to rise again quickly, entering a healthier or normal range at time t₆ and returning to an ideal range at time t₇. Without monitoring one or more secondary characteristics, the hypoglycemic event may not have been detected until the patient already had dangerously low blood sugar levels for an extended period of time, and rapid recovery may not have been possible. Thus, the insulin pump can actively analyze data coming from the primary sensor and the one or more secondary sensors to watch for an onset of a possible negative consequence that may not be as easily or quickly identifiable without being able to monitor multiple data sources at once. The pump can then react immediately to the possible event and either avoid the negative consequence entirely or, as is illustrated in FIG. 11, greatly reduce the negative effects.

The hierarchy between various sensors can be predefined; can be adaptable based on user input, such as providing input through the user interface 1085; can be adaptable based on a processor, an algorithm, any analyzed data, etc.; and/or can be adaptable through contact with remote computer systems, doctors, remote-care providers, etc. The insulin pump can also incorporate various functional components of the infusion pump system described in U.S. Patent Pub. No. 2009/0069787 entitled “Activity Sensing Techniques for an Infusion Pump System” published Mar. 12, 2009 and incorporated herein by reference in its entirety.

Additionally, the device indicator 1085, the user interface 1080, and the communications interface 1099 can allow the insulin pump to alert the user immediately before onset of hypoglycemia, such as by flashing, buzzing, speaking, providing a warning image, etc. They can thus allow the insulin pump to provide instructions to the user, such as to eat something or take a glucose tablet, and/or to send data indicative of gathered sensor data to a remote server for later detailed analysis or to prompt immediate review by a medical professional, who can then take appropriate actions to help the patient, such as calling the patient, sending messages through the insulin pump, alerting emergency services, etc.

While one possible hierarchy of sensors is discussed in relation to the insulin pump of FIG. 11, other hierarchies are possible. In general, a primary characteristic for a drug administration device can be a control measure, and secondary characteristic(s) or measures can be data taken from sources surrounding the primary characteristic and/or sources that can influence and/or be influenced by the primary source. For example, blood sugar level is a primary characteristic for insulin delivery, as shown in FIG. 11, but blood pressure is a primary characteristic for various blood pressure medications. Additionally, sources surrounding the primary source can take a variety of different forms, such as glucose level (for example, as measured by a micro needle application and/or sweat analysis); blood pressure (for example, as measured by various wearable cuffs); hydration (for example, as measured by perspiration level); heart rate and/or activity level (for example, as measured by various metabolic consumption rates, sitting or sedentary motion determined by elevation changes, various gyroscopes); EKG cycle; heart rate variability; various acute effects or activities to trigger measurement (such as sleep or sleep quality detection and/or meal detection, for example by analyzing one or more images of the patient, receiving input from the patient, etc.); discernment between eating and drinking; various long term effects to monitor any changes that might inform a new diagnoses or provide alerts to seek evaluation for any possible new conditions; core temperature; tremor detection; patient held/worn camera image analysis; time of day; digital calendar information; GPS outputs; device activity; any user interaction with the device; etc.

Additionally, numerous means for being aware of any surrounding situation during administration of the drug are possible beyond those discussed in relation to FIG. 11, providing a variety of types of situational awareness that one or more drug administration devices can use. As further examples, forms of cognitive analysis can be performed on the patient by combining small interactions with the patient and various automated sensors on or around the patient to determine cognitive effects of any drug dosage on the patient. Various measured reactions to drug dosages can also be analyzed, such as timing to a first effect, effect duration, magnitude of effect, etc. The insulin pump of FIG. 11 provides an example of ending continued application of a drug, however there are many other examples where such an action can be taken. For example, if a biologic or drug is being delivered on an ongoing basis, the plurality of sensors can allow detection of an onset of a complex biologic response to the biologic or drug, and a drug administration device can have the ability to affect, retard, or end the continued application of the biologic or drug. Thus, devices described herein can be configured to provide detection of and an automated response to collateral physiologic reactions to any continuous biologic introduction.

For example, injection reactions can be an issue for some biologics, especially when delivered through an IV given delivery times and the continuous administration. Thus, drug administration devices described herein can be configured to detect various onsets of injection reactions, such as through sensor(s), and consequently stop or slow down delivery of the drug. In at least some embodiments, drug administration devices described herein can be configured to deliver other medication(s) to stop, lessen, or counteract the drug injection reaction.

As another example, cytokine release syndrome is a form of systemic inflammatory response syndrome that can arise from an adverse effect of some monoclonal antibody drugs, as well as adoptive T-cell therapies. Once a drug administration device, or other system in communication with the device, detects pro- and anti-inflammatory components above a predetermined threshold in a patient, the drug administration device can be configured to reduce or stop the introduction of the treatment. In such an example, the device can also be configured to notify medical personnel or introduce a canceling agent to accelerate the reduction of the response. If the injection response is great enough as defined by predefined criteria, the drug administration device can be configured to automatically escalate its response from a passive indication or reduction of dosage to a more active warning notification or introductions of other active countermeasures. Even when medical intervention is required, such as requiring a patient to go into a hospital for emergency treatment, the drug administration devices described herein can be configured to use biometric data to detect changes in the patient's body, such as body temperature or heart rate, that typically proceed a serious effect. The drug administration device can be configured to notify the patient of the imminent effect to allow the patient to take preemptive action, such as taking medication at home, before then going into the hospital before one or more major side effects take place. This early warning can improve patient outcomes by reducing any negative consequences.

As yet another example, some drugs can cause drowsiness, dizziness, and/or other side effect that can adversely affect the patient's ability to drive and/or navigate safely from a hospital or other location of drug administration. When such a drug is administered to the patient at a location, such as a hospital or other medical care facility, from which the patient plans to drive home (or elsewhere), confirming that the patient is not experiencing any of the drug's possible side effects that can adversely affect the patient's ability to drive may help prevent the patient from unsafely driving post-drug administration. Once a drug administration device, or other system in communication with the device, detects any of the drug's possible side effects that can adversely affect the patient's ability to drive or navigate safely from the location of drug administration, the drug administration device, or other system in communication with the device, can be configured to notify medical personnel. The medical personnel may then help ensure that the patient does not leave the location of drug administration until the side effect(s) resolve and/or may allow the medical personnel to contact at the appropriate time the patient's preferred provider of transportation from the location of drug administration, e.g., a family member, a care provider, a taxi service, a ride share service, etc. The sensor data may also help evaluate the drug's side effects across a population of patients. In some embodiments, the patient may not be planning to drive after the drug administration, but being determined to not be experiencing any of the drug's possible side effects that can adversely affect the patient's ability to drive or navigate safely nevertheless helps medical personnel evaluate whether the patient is ready for release. For some drugs, such as esketamine, ketamine, and other controlled substances, the patient must wait at the site of drug administration for observation for a minimum amount of time after drug administration. The sensors may help ensure that at least at the end of the minimum amount of time the patient is not experiencing any of the drug's possible side effects that can adversely affect the patient's ability to drive, thereby helping medical personnel evaluate whether the patient is ready for release from observation.

In at least some implementations, drug delivery from a drug administration device is altered based on interaction between a drug administration device and an accessory, representing a cooperative or closed-loop relationship between the drug administration device and the accessory. The accessory can either be retained in or on the drug administration device or can be separate therefrom. The accessory includes a processor configured to receive data from at least one sensor of the drug administration device that is indicative of a patient's physiological characteristic and to control delivery of the drug from the drug administration device to the patient based on the received data. As such, dosages can be varied over time based on the sensor data and interaction with the accessory to allow for more personalized drug administration during each dose and over time, thus increasing the beneficial results of the drug by taking into consideration actual, present conditions of the patient. This functionality is similar to that discussed above with respect to FIGS. 9 and 10 except that the accessory, not the drug administration device, manages an algorithm controlling drug delivery from the drug administration device. The drug administration device may thus be less “smart” than the drug administration device 1000 of FIG. 9 and, consequently, be smaller and/or less expensive. Also, it is typically less expensive and/or easier for the patient to upgrade accessories and/or acquire new accessories than for the patient to upgrade the drug administration device or acquire a new drug administration device, so offloading processing and algorithm control to the accessory may extend the useful life of the drug administration device.

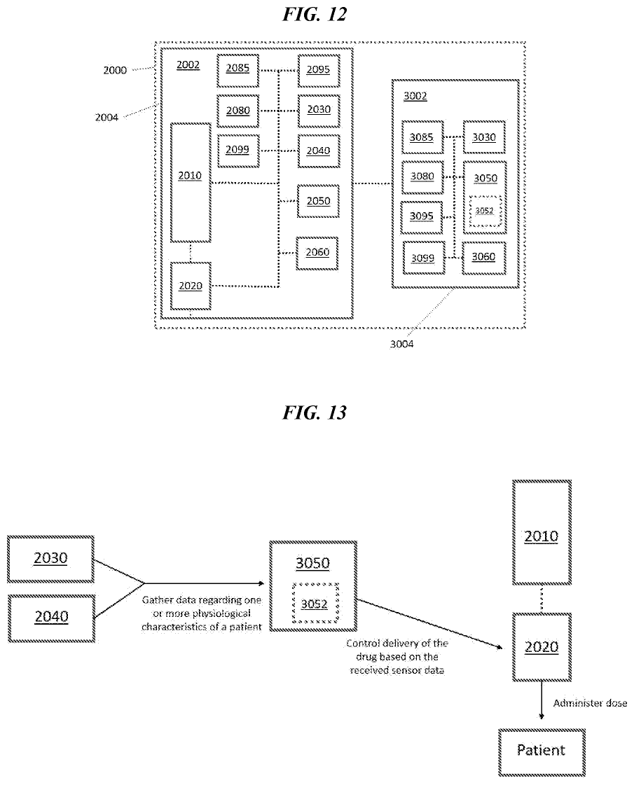

FIG. 12 illustrates one embodiment of a drug administration system 2000 including a universal drug administration device 2002 and an accessory 3002. The drug administration device 2002 in this illustrated embodiment includes a housing 2004, a drug holder 2010, a dispensing mechanism 2020, at least one sensor 2030, 2040, a memory 2050, a processor 2060, a user interface 2080, an indicator 2085, a power supply 2095, and a communications interface 2099. The accessory 3002 in this illustrated embodiment includes a housing 3004, at least one sensor 3030, a memory 3050 configured to store an algorithm 3052 therein, a user interface 3080, a device indicator 3085, a processor 3060, a power supply 3095, and a communications interface 3099. Additionally, similar to that mentioned above, a person skilled in the art will appreciate that each of the universal drug administration device 2002 and the accessory 3002 can be provided with a variety of the features described above, in a number of different combinations.

First and second sensors 2030, 2040 of the drug administration device 2002 are similar to the sensors 1030, 1040, 1045 of FIG. 9 discussed above. In an exemplary embodiment each sensor 1030, 1040 is configured to gather data regarding a physiological characteristic of the patient. For example, the sensed physiological characteristics can be any two or more of a reaction of the patient to the drug delivered thereto, blood sugar level, blood pressure, perspiration level, heart rate, respiratory rate, atmospheric sensing, angular rate, body orientation, MARG (magnetic, angular rate, and gravity), internal device sensing, blood oxygenation level, sun exposure, osmolality, piezoelectric skin measurements such as ultrasonic response changes, electrical parameter of dermis such as impedance, biosensing, sensing an enzyme, sensing an antibody, sensing a histamine, sensing a nucleic acid, any of the characteristics discussed for device 1000, air quality tracking, etc. Alternatively or in addition one or both of the sensors 1030, 1040 can be configured to gather data regarding a current of a motor used in delivering the drug. U.S. Patent Pub. No. 2002/0014951 entitled “Remote Control For A Hospital Bed” published Feb. 7, 2002, and U.S. Patent Pub. No. 2007/0251835 entitled “Subnetwork Synchronization And Variable Transmit Synchronization Techniques For A Wireless Medical Device Network” published Nov. 1, 2007, further discuss various sensors and are incorporated by reference herein in their entireties. The sensors 2030, 2040 can either monitor the same physiological parameter or monitor different physiological parameters. Monitoring the same parameter may allow for confirmation of a condition, while monitoring different parameters may allow for more characteristics associated with the patient to be considered for drug delivery. While two sensors are illustrated in FIG. 12, the device 2002 can include only one sensor, such as sensor 2030, or can include three or more sensors. Any additional sensors can operate similarly to sensors 2030, 2040 and can also monitor various physiological characteristics. One or both of the sensors 2030, 2040 can be a biosensor, e.g., a device that includes a biological component and a transducer and that is configured to sense a biological element such as an enzyme, an antibody, a histamine, a nucleic acid, etc. The sensors 2030, 2040 can operate together as a sensor array or a dual sensor. The sensors 2030, 2040 are configured to sense their respective physiological characteristic(s), and the memory 2060 is configured to store therein data as pluralities of data points defining and/or representing the sensed characteristic(s). The communications interface 2099 is configured to communicate data indicative of the sensed information to the accessory 3002, e.g., to the communications interface 3099 thereof to allow the received information to be stored in the memory 3050 and analyzed by the processor 3060 to control drug delivery from the device 2002 using the algorithm 3052.

The accessory's at least one sensor 3030 is configured to sense one or more characteristics associated with a patient. The one or more characteristics can be physiological characteristics and/or situational characteristics of the patient. Various different physiological characteristics can be monitored, such as heart rate, respiratory rate, blood pressure, perspiration level, etc. A number of different situational characteristics can be monitored, such as core temperature, time of day, date, patient activity level, altitude, GPS information, blood oxygenation level, sun exposure, osmolality, air quality, inflammatory response, one or more images and/or videos of the patient and/or an environment in which the patient is located (for example, to analyze food intake; to determine whether solid food or liquid is being consumed; to determine a location or activity of the patient; to determine a condition of the patient such as skin reaction, breathing, eye dilation, sedation, disassociation, voice characteristics such as tone and pitch; etc.), user-input data such as general well-being or a cycle time between flare ups of a particular ailment, etc. The at least one sensor 3030 is configured to sense the characteristic(s), with the memory 3050 storing gathered data as pluralities of data points defining and/or representing the sensed characteristic(s).

In at least some embodiments, one of the sensors 2030, 2040 of the drug administration device 2002 can in effect act as a primary sensor for the administration system 2000 that defines an operational range of closed loop control of the drug dosage and timing administration, and the at least one sensor 3030 of the accessory 3002 can act as a secondary sensor that provides patient feedback and is used to adjust the dosage amount, dosage timing, dosage location, etc. within the range defined by the primary sensor 2030.

The processor 3060 of the accessory 3002 is configured to control delivery of the drug from the device 2002 to a patient based on the sensed data received from the device 2002 and, in at least some embodiments, additionally or alternatively based on data gathered by the accessory's at least one sensor 3030. Similar to that discussed above, the processor 3060 is configured to control the drug delivery by changing at least one variable parameter of the algorithm 3052 such as adjusting at least one variable parameter for a dosage of the drug, a timing between doses of the drug, a location of delivery of the drug, a concentration of the dose, actuating a set number or a continuous number of discrete doses, actuating continuous dosage, actuating an initial bolus dose and then subsequent sporadic or repeating smaller doses as needed, terminating dosage, skipping one or more doses, etc. The data received at the accessory 3002 from the device 2002 is in the form of pluralities of data points defining the sensed physiological characteristic(s) data of the patient. The processor 3060 can be configured to change the at least one variable parameter of the algorithm 3052 during the delivery of a dose such that the algorithm 3052 is changed in real time with the delivery of the dose, which may accommodate real time sensed conditions, or the processor 3060 can be configured to change the at least one variable parameter of the algorithm 3052 before a start of the delivery of the dose, which may consume less memory and use fewer processing resources during algorithm 3052 execution than real time changing of the algorithm 3052.

The processor 3060 is configured to control drug delivery from the device 2002 through any of a variety of different mechanisms, such as by transmitting a command to the device 2002 using the communications interfaces 2099, 3099 with the device's processor 2060 executing the command to cause drug delivery (e.g., by providing a plurality of data points defining one or more instructions to the device 2002). Instead of the algorithm 3052 being stored at the accessory 3002, the algorithm for controlling drug delivery from the device 2002 can be stored at the device 2002, e.g., in the memory 2050 thereof. Similar to that discussed above, the accessory 3002 can be configured to cause at least one variable parameter of the algorithm stored at the device 2002 to be changed based on the data gathered by the device's at least one sensor 2030, 2040 and/or based on the data gathered by the accessory's at least one senor 3030, e.g., by transmitting a command to the device 2002 using the communications interfaces 2099, 3099 with the device's processor 3060 executing the command to change the at least one variable parameter as instructed. The algorithm being stored at the device 2002 instead of the accessory 3002 may help ensure that drug delivery occurs since delivery is controlled locally and can occur even if communication is broken between the device 2002 and accessory 3002, e.g., because the device 2002 and accessory 3002 are out of wireless communication range of one another, because of a network system problem, because of power loss at the accessory 3002, etc.

FIG. 13 illustrates an embodiment of use of the drug administration device 2002 and accessory 3002. The use is similar to that discussed above with respect to FIG. 10 except that the processor 3050 of the accessory 3002 is involved in data analysis and control of dose delivery.

Various delivery types can be used from the drug administration device 2002, such as bolus or basal deliveries. For example, based on the severity of a hyper- or hypoglycemic reaction, a large bolus delivery can be used to prevent a body of a patient from further degrading. For another example, an automated system can adjust basal insulin target dose sizes based on a long term tracking of blood sugar, continuously adjust around a target basal level based on continuous monitoring, and then introduce bolus levels only for severe adjustments.

In at least some embodiments, the drug administration device 2002 is a smart drug administration device that performs some analysis on its own, with the accessory 3002 providing additional smart functionality to help improve dosage effectiveness, safety, and/or accuracy. For example, a smart drug administration device can be a smart insulin pump, such as Medtronic's MiniMed 670G, that allows for blood sugar detection such that the pump tracks a patient's continuous glucose level while adjusting for proximity between pump and sensor. For another example, a smart drug administration device can be a glucagon delivery device that treats hypoglycemia severity (defined by a blood sugar under 70) by injecting glucagon to raise glucose leveled to the desired basal level. For yet another example, a smart drug administration device can be an insulin delivery device that continuously adjusts an amount of insulin it delivers from one minute to the next based upon readings from the continuous glucose monitor.

The accessory 3002 can take a variety of different forms. The accessory 3002 can be used solely for helping to control drug delivery from the drug administration device 2002. Alternatively, the accessory 3002 can have an additional purpose that is different than that of drug delivery from the drug administration device 2002 and thus be multi-functional.

Embodiments of accessories include ear wigs, ear pieces, smart watches, fingernail sensors, digital collection patches (with or without direct skin contact), augmented reality or smart glasses, implantable or ingestible components, headbands, digitally connected devices to communicate weight, worn cameras (such as in smart glasses), carried cameras (such as in smartphones, mobile tablets, etc.), handheld diabetes management devices, smart mobility aid devices, tracking and monitoring mechanisms, etc. U.S. Patent Pub. No. 2014/0081659 entitled “Systems And Methods For Surgical And Interventional Planning, Support, Post-Operative Follow-Up, And Functional Recovery Tracking” published Mar. 20, 2014 describes various embodiments of tracking and monitoring mechanisms and is incorporated by reference herein in its entirety. U.S. Patent Pub. No. 2012/0095318 entitled “Handheld Diabetes Management Device With Bolus Calculator” published Apr. 19, 2012, which is incorporated by reference herein in its entirety, describes embodiments of handheld diabetes management devices. U.S. Patent Pub. No. 2017/0172462 entitled “Multi-Functional Smart Mobility Aid Devices And Methods Of Use” filed on Jun. 22, 2017, which is incorporated by reference herein in its entirety, describes embodiments of smart mobility aid devices.

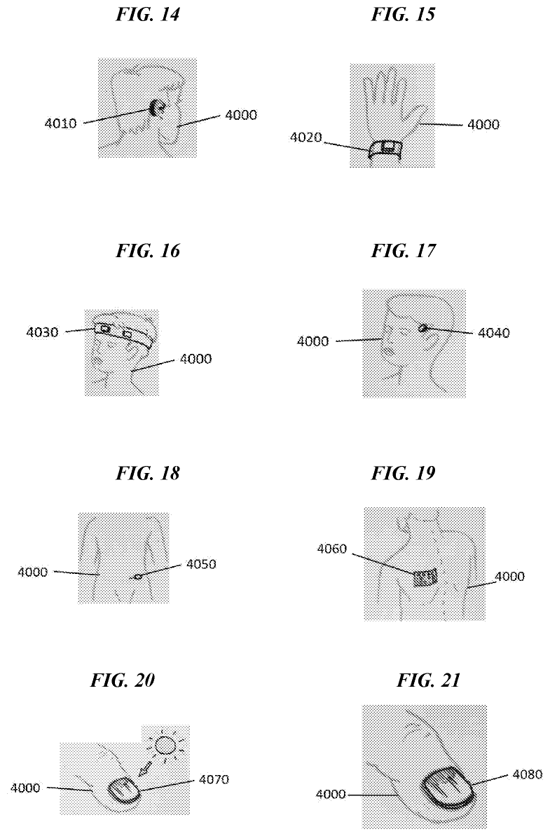

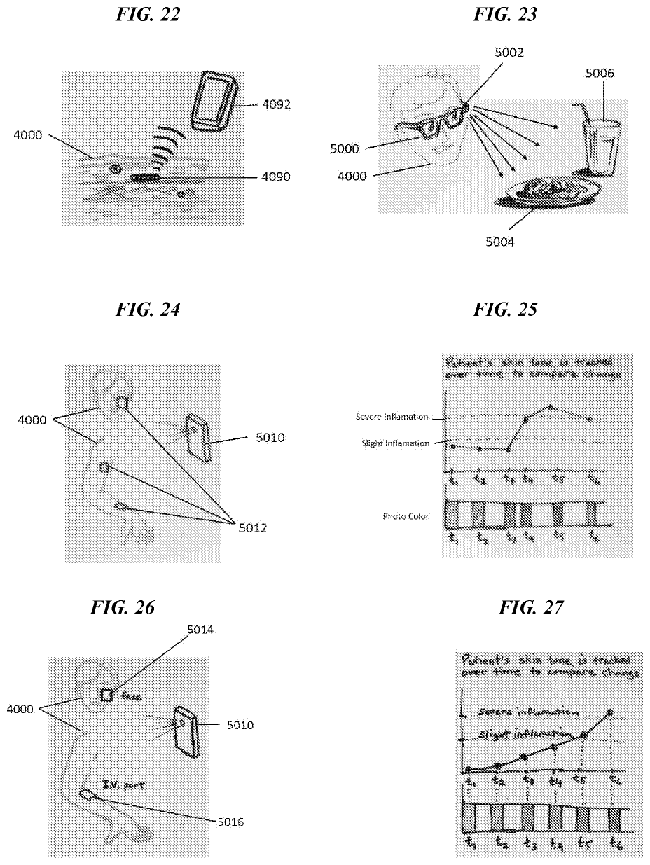

FIGS. 14-24, 26, and 28 illustrate various embodiments of accessories that can be used as the accessory 3002. FIG. 14 illustrates an embodiment of an accessory 4010 in the form of an ear piece configured to be worn by a patient 4000 around their right ear or left ear. FIG. 15 illustrates another embodiment of an accessory 4020 in the form of a wristband or smartwatch configured to be worn on the right or left wrist of the patient 4000. FIGS. 16 and 17 illustrate embodiments of accessories 4030, 4040 configured to be worn on the head of the patient 4000. Accessory 4030 is a headband worn around the patient's head, and accessory 4040 is a device that is attached directly to skin of the patient's head, such as by adhesive. FIGS. 18 and 19 illustrate embodiments of accessories 4050, 4060 configured to be worn on the body of the patient 4000. Accessory 4050 is an abdomen patch or device that is placed directly on the patient's abdominal skin, and accessory 4060 is a patch or device that is attached to skin of the patient's back. Both accessories 4050, 4060 can be attached in a variety of ways, such as through use of adhesive. FIGS. 20 and 21 illustrate embodiments of accessories 4070, 4080 that are wearable fingernail sensors configured to be worn on one or more fingernails of the patient 4000. Accessory 4070 is a photo or light sensing wearable fingernail sensor configured to detect presence of various types of light, such as UV sunlight, and accessory 4080 is a chemical-sensing wearable fingernail sensor configured to detect the presence and/or concentration of various chemicals. FIG. 22 illustrates an embodiment of an accessory 4090 configured to be implanted in and/or ingested by a patient 4000. FIG. 22 also illustrates an embodiment of a drug administration device 4092, similar to the drug administration device 2002 of FIG. 12, that is located outside of the patient and configured to be in electronic communication with the implanted/ingested accessory 4090.

As mentioned above, image analysis can be used for capturing data in the environment around the patient. For example, as will be appreciated by a person skilled in the art, image analysis can be used for meal detection, such as for confirmation that a meal is occurring; analysis of a meal itself such as volume or carbohydrate, protein, and fat content; image analysis of skin tone, injection site, and/or other anatomic structure to determine redness, inflammation, and/or other reaction; calculation of dosage amounts for drugs that are administered based on body area (for example, mg/m²) such that images of the body of a patient can be used to calculate a dose in addition to any patient inputs such as weight, age, etc.; image analysis to provide relevant drug information through an interconnection between an image taken by a patient and any smart digital patient device that would allow the device to provide user information on the medication, dosage, timing, function, etc. U.S. Patent Pub. No. 2012/0330684 entitled “Medication Verification And Dispensing” published Dec. 27, 2012, which is incorporated by reference herein in its entirety, further describes image capturing devices. In response to detecting a meal, the accessory can be configured to adjust drug delivery from the drug administration device in communication with the accessory.

FIG. 23 illustrates an embodiment of an accessory 5000 in the form of smart glasses with a camera 5002 built therein. The patient 4000 can wear the accessory 5000 like a normal pair of glasses, however the accessory 5000, e.g., a processor thereof, is configured to analyze images captured by the camera 5002 to visually identify a variety of types of information about a meal 5004 and/or a drink 5006 that the patient 4000 is consuming, such as food type, food amount, amount of food remaining on plate, etc. U.S. Patent Pub. No. 2011/0295337 entitled “Systems and Methods For Regulating Metabolic Hormone Producing Tissue” filed on Dec. 1, 2011, U.S. Pat. No. 8,696,616 entitled “Obesity Therapy And Heart Rate Variability” issued Apr. 15, 2014, U.S. Pat. No. 9,427,580 entitled “Devices And Methods For The Treatment Of Metabolic Disorders” issued Aug. 30, 2016, and U.S. Pat. No. 9,168,000 entitled “Meal Detection Devices And Methods” issued Oct. 27, 2015, which are incorporated by reference herein in their entireties, further describe identifying types of information about a meal and/or a drink. Detecting occurrences of eating/drinking with certainty is important for safety, efficacy, and cost, and can be combined with sensed information through situational awareness, as discussed above regarding device 1000, to increase the accuracy of meal detection methods described herein.

An accessory configured for meal detection, such as the accessory 5000, can be used in a variety of different situations. For example, activation of brown adipose tissue (BAT) is known to increase metabolic activity. BAT activity results in thermogenesis, which can be measured with a temperature probe of some sort. Activating BAT can have a large metabolic impact when associated with a meal. Therefore, the accessory configured for meal detection can be used to detect a meal, and the detected meal can trigger a release of a drug dose used for BAT activation. Confirmation of the activation can then be determined with a temperature probe that is worn adjacent to a BAT depot. For example, U.S. Pat. No. 9,610,429 entitled “Methods And Devices For Activating Brown Adipose Tissue With Targeted Substance Delivery” issued Apr. 4, 2017 and U.S. Pat. No. 9,381,219 entitled “Brown Adipocyte Modification” issued Jul. 5, 2016, which are incorporated by reference herein in their entireties, further describe drug based activation means. U.S. Pat. No. 8,812,100 entitled “Device And Method For Self-Positioning Of A Stimulation Device To Activate Brown Adipose Tissue Depot In A Supraclavicular Fossa Region” issued Aug. 19, 2014 and incorporated by reference herein in its entirety, further describes temperature probes.

As mentioned above, different sensor configurations and interactions can be used to produce primary and secondary measurements of physiological characteristic(s) of the patient, both in the drug administration device and/or the accessory. For example, sensors used can be modular sensor arrays, configurable sensor arrays, dual sensors that provide interactive sensing, dual cooperative remote sensing arrays, etc. In such examples, one or more sensor(s) configured to detect a physiologic response to drug administration in the patient can be positioned remotely to an injection site to prevent drug administration itself from interfering with the result. In addition, a second sensor array can be positioned close to the injection site and/or the drug administration device in order to determine acute local reaction and verify that the drug administration device has operated correctly.

FIGS. 24 and 26 illustrate an embodiment of an accessory 5010 including a camera configured to gather images of the patient's eyes and/or the skin of the patient 4000 at various points on the patient's body. The accessory 5010 is configured to monitor the patient's eyes and/or skin tone, either at a single point or, as illustrated in FIG. 25 (for analysis of data gathered as shown in FIG. 24) and FIG. 27 (for analysis of data gathered as shown in FIG. 26), over time to track any possible reactions to a drug (e.g., sleep, drowsiness, etc.) and/or to watch for any inflammation (either caused by administration of the drug or caused by a secondary source and for which the drug is being administered to treat) and, in response to detecting a drug reaction and/or inflammation, adjust drug delivery from the drug administration device in communication with the accessory 5010. FIGS. 25 and 27 illustrate times t₁, t₂, t₃, t₄, t₅, and t₆ and a corresponding photo color chart of the patient's skin tone at each point in time (shown in grayscale in FIGS. 25 and 27). The broken lines traced on the graphs in FIGS. 25 and 27 represent slight inflammation and severe inflammation such that image analysis (either electronically by the system or manually by a care professional) allows tracking of inflammation and alerts, notification, pre-emptive action, responsive action, etc. in response to skin tone passing over the slight and/or the severe inflammation. FIG. 24 illustrates taking images at random points 5012 on the patient 4000. FIG. 26 illustrates taking images of the patient's face 5014 and at the point of administration of the drug in the form of an IV port 5016. The point of administration and the face 5014 of the patient 4000 can be useful areas to monitor for any adverse or beneficial reactions to an administered drug because the site of administration (the IV port 5016) is the first point of contact between the drug and the patient 4000 and human faces can be expressive of possible adverse reactions such as allergic reactions, etc.

Although FIGS. 24 and 26 show the camera as being part of a smartphone, the accessory configured to gather images can be a device other than a smartphone, such as a mobile tablet, a smartwatch, etc. Additionally, although the same accessory 5010 is shown gathering the images in FIGS. 24 and 26, two different accessories can gather the data of FIGS. 24 and 26.

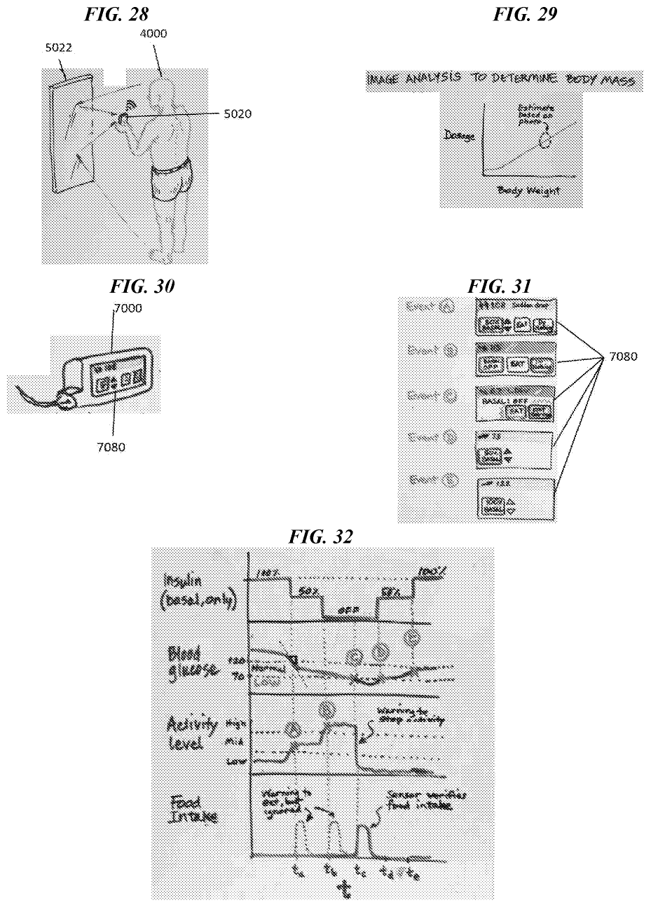

FIG. 28 illustrates an embodiment of an accessory 5020 including a camera configured to gather images of the body of the patient 4000, for example in a mirror 5022. The accessory 5020 is a smartphone in this illustrated embodiment, but as discussed above, it can be another type of accessory configured to gather images. The accessory 5020 is configured to estimate the body weight of the patient 4000 based on one or more of the gathered images and to use the estimated body weight in changing the algorithm for drug delivery from a drug administration device associated with the patient 4000. Dosages of various medications can be dependent on body weight, and one or more images of the full body of the patient 4000 can allow the accessory 5020 and a corresponding drug administration device a simple way to provide correct dosages based on the estimated body weight of the patient 4000. FIG. 29 illustrates an embodiment of a graph correlating estimated body weight and dosage that the accessory 5020 is configured to use in adjusting dose delivery based on estimated body weight.

In at least some implementations, drug delivery from a drug administration device is altered based on an awareness of a status of a patient, such as altering drug delivery based on at least one physiological characteristic of the patient and on at least one related physical characteristic of the patient. These implementations are similar to those discussed above with respect to drug delivery being altered based on one or more characteristics associated with the patient that are determined based on situational awareness of the patient except that the characteristics associated with the patient are determined based on at least one physiological characteristic of the patient and on at least one related physical characteristic of the patient. The implementations that take into account physiological characteristic(s) and physical characteristic(s) allow dosages to be varied based on a status of the patient, represented by the physiological and physical characteristics of the patient, to allow for personalized care, an adaptive drug administration process to improve patient care, and/or automatic dose adjustment to increase successful drug use by patients. Various different physiological characteristics of the patient can be monitored, such as body temperature, heart rate, blood sugar level, blood pressure, perspiration level, etc. Various different physical characteristics of the patient can be monitored, such as temperature, metabolic demand, cognitive function, metabolic demand such as measured using at least one of food intake and BMR (basal metabolic rate), weight, one or more images and/or videos of the patient and/or an environment in which the patient is located (for example, to analyze food intake, to determine whether solid food or liquid is being consumed, to determine a location or activity of the patient, to determine a condition of the patient such as skin reaction, etc.), etc. Various different physical characteristics of the patient's environment can be monitored, such as atmospheric contaminant percentage, environmental temperature, etc.

Using the universal drug administration device 500 of FIG. 5B by way of example, the memory 97 can have stored therein the algorithm executable to administer a dose of drug to a patient, and the processor 96 can be configured to execute the algorithm to control delivery of a dose of the drug dispensed by the dispensing mechanism 20. The processor 96 can also be configured to use physiological data representative of at least one physiological characteristic of the patient and physical data representative of at least one physical characteristic of the patient to change at least one variable parameter of the algorithm such that a dose delivered subsequent to the changing of the at least one variable parameter will be controlled by execution of the changed algorithm. As mentioned above, a person skilled in the art will appreciate that the universal drug administration device 500 can be provided with a variety of the optional features described above, in a number of different combinations, e.g., not include any of the sensors 92, 94, 98 not being used by the processor 96 to change variable parameter(s) of the algorithm (and instead include sensor(s) to gather the needed physical and physiological characteristic data), not include packaging 35, not include user interface 80, etc. The processor 96 can be configured to change the at least one variable parameter of the algorithm during the delivery of a dose such that the algorithm is changed in real time with the delivery of the dose, which may accommodate real time sensed conditions, or the processor 96 can be configured to change the at least one variable parameter of the algorithm before a start of the delivery of the dose, which may consume less memory and use fewer processing resources during algorithm execution than real time changing of the algorithm.

Using the universal drug administration device 1000 of FIG. 9 by way of another example, the use is similar to that discussed above regarding FIGS. 9 and 10. The memory 1050 has stored therein the algorithm 1052, and the processor 1060 is configured to execute the algorithm 1052 to control delivery of a dose of the drug dispensed by the dispensing mechanism 1020. The processor 1060 is also configured to use physical characteristic data and physiological characteristic data gathered by at least two of the sensors 1030, 1040, 1045 to change at least one variable parameter of the algorithm 1052 such that a dose delivered subsequent to the changing of the at least one variable parameter will be controlled by execution of the changed algorithm 1052. As mentioned above, a person skilled in the art will appreciate that the universal drug administration device 1000 can be provided with a variety of the optional features described above, in a number of different combinations, e.g., not include any of the sensors 1030, 1040, 1045 not being used by the processor 1060 to change variable parameter(s) of the algorithm 1052 (and instead include sensor(s) to gather the needed physical and physiological characteristic data), not include user interface 1080, etc. The processor 1060 can be configured to change the at least one variable parameter of the algorithm during the delivery of a dose such that the algorithm is changed in real time with the delivery of the dose, which may accommodate real time sensed conditions, or the processor 1060 can be configured to change the at least one variable parameter of the algorithm before a start of the delivery of the dose, which may consume less memory and use fewer processing resources during algorithm execution than real time changing of the algorithm.

In at least some embodiments, the at least one physiologic characteristic is directly tied to the treatment being administered by the drug administration device, and the device is configured to use local or immediate processing to determine and adjust dosage to compensate for or overcome one or more current physical characteristic(s) being sensed. In such embodiments, as discussed further below, the at least one physiological characteristic is the primary characteristic that defines a range of responses of the drug administration device and the at least one variable parameter, and the one or more physical characteristics are secondary characteristics used to fine-tune or influence the device's dosage by changing the at least one variable parameter.

The implementations in which drug delivery from a drug administration device is altered based on an awareness of a status of a patient may allow for detection of any number of philological and/or physical characteristics. For example, detection of a physical characteristic of activity level, metabolism, and/or metabolic level can influence adjustment of dosage based on increased physiological demand. Generally, metabolism and metabolic rates are a blend between total energy expenditure and the energy balance between caloric intake and loss. Precise measurements of activity, caloric intake, fecal caloric output, oxygen consumption/CO₂ generation, etc. can thus be used to inform metabolic activity. In such embodiments, metabolic activity can be the measured physiological characteristic, and one or more physical characteristics, such as activity, caloric intake, fecal caloric output, oxygen consumption/CO₂ generation, etc. can be measured to guide adjustment of drug administration based on the measured philological characteristic. In other embodiments, a combination of a variety of different measurements, such as activity measures, food intake measures, BMR, a physiologic measure such as body temperature or change in temperature, environmental temperature, and/or heart rate can be used as various approximations for real-time metabolic rates of a patient rather than attempting a more direct measurement. Metabolic rates are further discussed in, for example, Lam Y Y and Ravussin, E., “Analysis of energy metabolism in humans: A review of methodologies,” Mol Metab. 2016 November; 5(11): 1057-1071 (available at <https://www.ncbi.nlm.nih.gov/pmc/articles/PMC5081410/>), which is hereby incorporated by reference in its entirety.

Drug delivery adjustment in implementations in which drug delivery from a drug administration device is altered based on an awareness of a status of a patient can be fully automatic, partially automatic and partially manual, or fully manual, partially automatic or fully automatic devices. Some degree of automatic dosage adjustment can be especially beneficial in situations when patients may have difficulty applying any recommended changes themselves caused by various patient circumstances, environmental cues, and/or physiological cues. For example, patient capacity and/or competency levels can be used as one or more means to determine between a fully automated response by the drug administration device and/or varying degrees of user partial control over dosage adjustment. For example, patients may normally be able to interact with the device(s) safely, but during various emergency situations, the patient's abilities may be impaired and automatic action may be required.

FIG. 30 illustrates one embodiment of a drug administration device 7000 configured to have drug delivery therefrom altered based on an awareness of a status of a patient. The device 7000 is a partially-automatic insulin pump and includes a user interface 7080 that is similar to the user interface 1080 and the device indicator 1085 of FIG. 9. FIG. 31 also illustrates five exemplary views of the user interface 7080 showing information associated with five different events A, B, C, D, and E, discussed further below. As shown in FIG. 32, the device 7000 is configured to measure blood glucose as the physiological characteristic, to measure activity level and food intake as two different physical characteristics, and to vary delivered insulin level as the at least one variable parameter. FIG. 32 identifies times of the Events A, B, C, D, and E of FIG. 31. The device 7000 in this illustrated embodiment is configured to provide three types of possible interaction with a patient using the device 7000: a fully automatic mode with no manual overrides but with provided alerts/recommendations, an automatic mode that provides alerts/recommendations to the patient and accepts user input, and an automatic mode that provides no alerts/recommendations.

The Events A, B, C, D, E are determined based on one or more of the physiological characteristic and/or physical characteristic sensed data. Occurrence of the Events A, B, C, D, E is configured to prompt alerts/recommendations to be provided to the patient via the user interface 7080 as shown in FIG. 31 and/or to prompt automatic action by the device 7000. For example, at Event A at time tₐ (see FIG. 32), increased activity level was detected, such as due to exercising, which caused a drop in the patient's blood glucose level from above 120 mg/dL into a normal range between 120 mg/dL and 70 mg/dL. While this drop does not place the patient at a dangerous low for blood glucose levels, the slope of the decline suggests that the blood glucose level of the patient may continue to decrease, which would place the blood glucose level at a dangerous level below 70 mg/dL. This drop triggered a minor alarm and a recommendation by the device 7000 on the user interface 7080 (Event A in FIG. 31) to choose between a reduction in the basal level of insulin being delivered, eating something, or ignore the recommendation. The user chose a 50% reduction in the basal level of insulin, as shown between times tₐ and t_{b}. The food intake graph in FIG. 32 indicates suggested eating by the broken curve at tₐ, but the recommendation was not taken.

At time t_{b} and Event B (see Event B in FIG. 32), an increase in physical activity was again detected, causing a significant drop in blood glucose levels toward a potentially low level of 70 mg/dL. This drop triggered a major alarm and a recommendation on the user interface 7080 (Event B in FIG. 31) to eat something or ignore. The patient ignored the recommendation. However, the device 7000 is partially automated and, as such, the device 7000 determined to discontinue delivering insulin at time t_{b}. In other embodiments, the device might ask the patient and/or a doctor what action to take regarding insulin delivery.

At Event C and time t_{c} (see FIG. 32), the device 7000 provided a major alarm and a recommendation on the user interface 7080 (Event C in FIG. 31) for eating something or stopping all physical activity because the previous activity level had not decreased, which caused blood glucose levels to move into a dangerously low range of 70 mg/dL. The device 7000 kept insulin delivery turned off automatically, and the device 7000 continued to sound the alarm until food intake was detected and/or all activity stopped. At time t_{c}, the patient did eat food, represented by a solid line on the food intake graph of FIG. 32.

At Event D and time t_{d} (see FIG. 32), a minor alarm sounded to alert the patient that the device 7000 is resuming insulin delivery at a basal level of 50% (see Event D in FIG. 31). The patient has the ability to adjust the delivery percentage if desired. Blood glucose levels enter a normal range at t_{d} and enter a higher range at tₑ (see FIG. 32), thus causing the device 7000 to provide one last notification (see Event E in FIG. 31) that insulin levels are returning to 100% basal levels automatically, however the patient can make changes if desired. When the patient's blood glucose levels drop dangerously low, the patient's ability to think clearly may be impaired, at which point the device 7000 can take various actions automatically to provide as much assistance to the patient as possible.

Thus, drug administration devices in at least some embodiments herein can be partially automatic, allowing patient and/or doctor control or override of automatic actions in various situations while providing automatic actions that cannot be overridden by a patient in various emergency situations. The notifications in at least some embodiments can have priorities or degrees to them, becoming more insistent as a situation becomes more dangerous for the patient. With certain drug administration devices, care providers and/or emergency personnel may be automatically alerted when certain major alarms are sounded. Additionally, risk-based assessments of treatment can become more aggressive over time if a patient fails to take appropriate de-escalation actions and/or the drug administration device detects an apparent lack of patient ability to help themselves.

A variety of other patient circumstances may lend themselves to some degree of partial or full automation for dosage adjustments. For example, a pediatric patient may not yet understand or be able to manipulate the drug administration device in a safe way, the patient may suffer from dementia while having a disease requiring an injectable or oral medication and cannot understand the drug administration device, patients may suffer from various mental or psychiatric disorders that affect their ability to use the drug administration device and/or to perceive gradual symptom escalation, patients may have a hard time administering the drug and need to rely on automatic actions by the drug administration device, etc. In at least some situations, recommended treatments can allow for drug administration device adjustments that simply make more sense to automate to help with compliance, such as with injectables that may be various forms of pumps or other drug administration devices almost always worn by a patient. For example, various diabetes side effects can increase a difficulty or inhibit a patient's ability to administer an adjusted dosage due to age, dexterity, onset of various complicating situations (such as hyper- or hypoglycemia), etc.

Similar to the accessory 5020 discussed above, at least some embodiments of drug administration devices can be configured to automatically adjust dosage over time based on a patient's weight, such as for a pediatric patient since weight tends to fluctuate more for pediatric patients as they are in the process of growing. Weight measurements can be taken in a variety of ways, such as from a scale in the home that automatically communicates the measured weight to the drug administration device or by analyzing images as discussed above. Warnings of weight change can also be provided prior to any change to a dosage actually occurring. In some situations, independent verification and/or approval by a patient's care provider may be required before initiating any change. For example, parents and/or doctors can report a pediatric patient's weight when possible, and the dosing algorithm for the patient can then update automatically with the new weight information. Any changes can be made directly on the device and/or can be made remotely.

In at least some embodiments in which drug delivery from a drug administration device is altered based on an awareness of a status of a patient, such as altering drug delivery based on at least one physiological characteristic of the patient and on at least one related physical characteristic of the patient, situational awareness of the patient can also be considered in altering the drug delivery, similar to that discussed above, for example, with respect to the drug administration device 1000 of FIG. 9. For example, additionally taking into account situational awareness can allow a drug administration device to refine sensed data, mitigate errors, eliminate or reduce inaccurate outliers, and/or otherwise selectively affect the drug administration device to improve the device's understanding of the physiological reactions of the patient leading up to or after a dosage administration. Thus, one or more sensors can act as adaptive sensing arrays based on a situational awareness of the patient and/or the drug administration device.

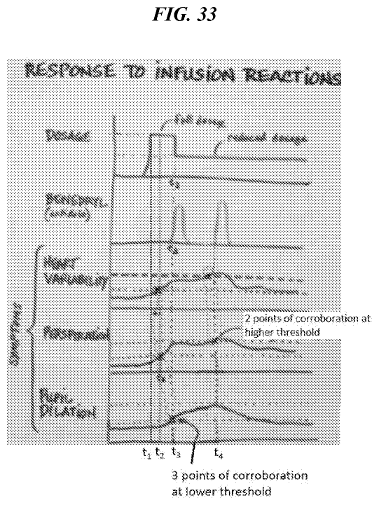

FIG. 33 illustrates an embodiment of an adverse reaction to a drug administration device in the form of an infusion device that delivers a dosage of a drug to a patient at time t₁. Along with various physiological characteristic(s) of the patient and physical characteristic(s) of the patient that the drug administration device tracks to determine appropriate dosages of the drug, the drug administration device also monitors for an allergic reaction to the drug by the patient by measuring physical characteristics and physiological characteristics that can indicate an adverse reaction, such as heart rate variability, perspiration, and/or pupil dilation. At time t₂, heart rate variability, perspiration, and pupil dilation all increase measurably, crossing over minor warning thresholds as illustrated and suggesting a possible adverse reaction to the drug. Thus, at time t₃, the dosage of the drug is reduced and a bolus of a medication, e.g., Benadryl, is administered by the device at a lower dosage to counteract the patient reaction. At time t₄, heart rate variability, perspiration, and pupil dilation all continue to increase, and heart rate variability and perspiration both cross over major warning thresholds. The drug administration device thus delivers a second larger bolus of the medication, at which point heart rate variability, perspiration, and pupil dilation all begin to decrease to more normal or baseline values. At this point in time, the patient's reaction is under control, so the original drug can continue to be delivered at the reduced dosage.

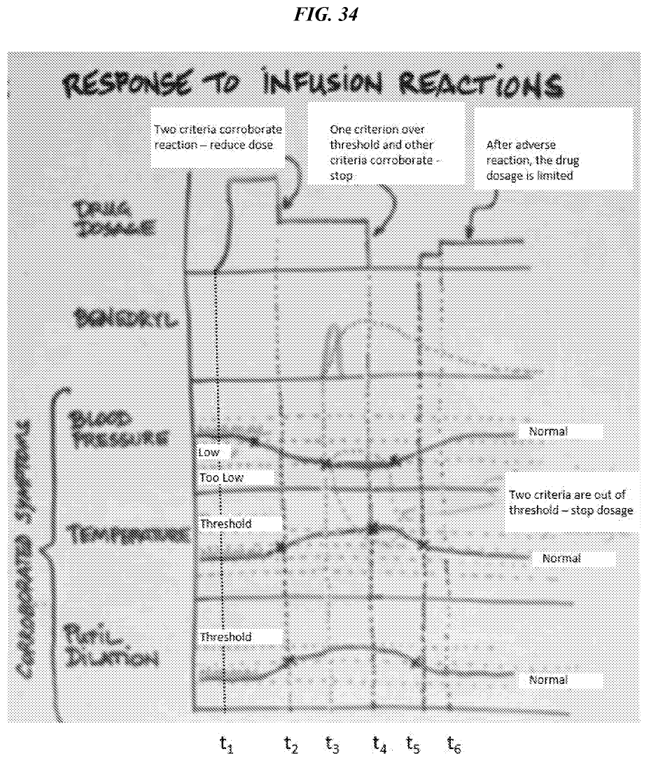

If the patient does not react effectively to drug administration, dosages of the original drug can be terminated entirely and restated at lower, stepped values only once the various symptoms of the adverse reaction have abated. For example, FIG. 34 illustrates a graph similar to the graph of FIG. 33 and shows measured blood pressure, temperature, and pupil dilation. At time t₁, a dosage of the drug begins to be administered to the patient. At time t₂, temperature and pupil dilation cross warning thresholds to indicate a possible adverse reaction, and the dosage is reduced. At time t₃, a dose of the medication is administered because blood pressure has gone down but both temperature and pupil dilation have continued to increase. At time t₄, pupil dilation is still elevated while temperature has crossed over a major threshold and blood pressure has decreased to such an extent that it has crossed over a lower threshold and is now too low. As such, drug dosage is stopped entirely. At t₅, the blood pressure, temperature, and pupil dilation have begun to return to normal levels, so the drug is again administered to the patient. However, the dosage is further reduced. At time t₆ when there has not been a significant increase in adverse reaction indications from the blood pressure, temperature, and pupil dilation, the dosage is again increased slightly and maintained at a lower level for the time being.

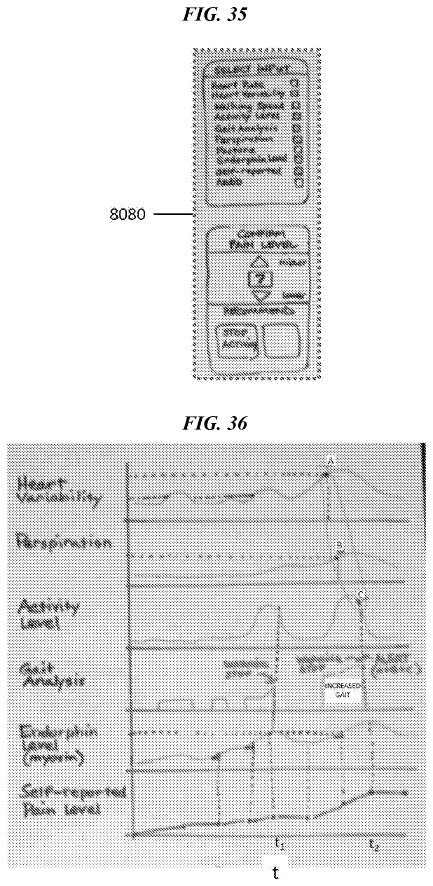

In at least some embodiments, patients can choose or opt in to monitoring from one or more sensors in a drug administration device. For example, FIG. 35 illustrates one embodiment of a user interface 8080 of a drug administration device that allows a patient, either on the device itself or via other means such as a patient app associated with the device, to opt into monitoring using one or more sensors. As the sensors monitor the patient as shown in FIG. 36, the sensors can detect possible or likely early onset of joint pain, such as at t₁ and t₂. For example, a minor warning might be prompted at time t₁ when readings from an activity level sensor and a gate analysis sensor indicate that physical activity may be too much for the patient and continued activity will prompt joint pain. As the patient continues activity, time point A on FIG. 38 indicates crossing over a major threshold for heart rate variability, time point B indicates crossing over a major threshold for perspiration, and time point C indicates crossing over a major threshold for activity level, at which point an alert is sounded when all three conditions A, B, C are met at time t₂ to indicate possible onset of severe joint pain. At this point, the device and/or the patient app can prompt the patient to enter a pain score, for example by using the user interface 8080, so that the device and any treating care provider can be better informed of the results of treatment. The situational awareness measurements discussed herein can be incorporated into any of the devices above to provide increased understanding of treatments and results and increased personalization of care.

As discussed above, some form of food intake and/or meal detection can be important when providing recommendations to a patient and when adjusting dosages. While some exemplary meal detection approaches are discussed above, such as image analysis, various drug administration devices can also use combinations of inputs from various physiological and/or physical sensors to confirm that a meal event has occurred and that it is significant enough to trigger a desired response in the patient. For example, analysis of heart rate variability (HRV), image analysis, gastric pH, a LINX Reflux Management device, etc. can each be used independently or in some combination to provide a more accurate detection of meal consumption. Providing various redundant measures may help minimize errors in meal detection.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

What is claimed is:

1. A drug administration device, comprising:
a drug holder configured to retain a drug therein;
a first sensor configured to gather data regarding a first characteristic associated with a patient;
a second sensor configured to gather data regarding a second characteristic associated with the patient;
a memory configured to store therein an algorithm including at least one variable parameter; and
a processor configured to:
control delivery of a first dose of the drug from the drug holder to the patient by executing the algorithm,
change the at least one variable parameter of the algorithm stored in the memory based on the data gathered by the first sensor and data gathered by the second sensor, and
after changing the at least one variable parameter, control delivery of a second dose of the drug from the drug holder to the patient by executing the algorithm.

2. The device of claim 1, wherein the processor is also configured to automatically control delivery of the doses according to a predetermined schedule of dosing for the patient.

3. The device of claim 1, further comprising at least one additional sensor, each sensor configured to gather data regarding a different characteristic;
wherein the processor also being configured to change the at least one variable parameter of the algorithm stored in the memory based on the data gathered by the at least one additional sensor.

4. The device of claim 1, wherein the processor is further configured to consider the data gathered by each of the first and second sensors in a hierarchy in changing the at least one variable parameter.

5. The device of claim 1, wherein the first characteristic is a physiological characteristic of the patient; and
the second characteristic is a situational characteristic of the patient.

6. The device of claim 1, wherein the first characteristic is one of blood sugar level, blood pressure, perspiration level, and heart rate; and
the second characteristic is at least one of core temperature, tremor detection, time of day, date, patient activity level, blood pressure, metabolic rate, altitude, temperature of the drug, viscosity of the drug, GPS information, angular rate, current of a motor used in delivering the drug, blood oxygenation level, sun exposure, osmolality, and air quality.

7. The device of claim 1, wherein the second sensor is configured to gather data by capturing an image of at least one of the patient and an environment in which the patient is located; and
the processor is configured to analyze the image to determine at least one of whether food intake occurred and skin reaction to the drug.

8. The device of claim 1, wherein the processor is also configured to, based on at least one of the data gathered by the first sensor and the data gathered by the second sensor, cause a device operation prevention mechanism to move from an unlocked state, in which the device operation prevention mechanism allows delivery of the drug to a user, to a locked state, in which the device operation prevention mechanism prevents delivery of the drug to the user.

9. The device of claim 8, wherein the drug administration device comprises one of an injection device, a nasal spray device, and an inhaler.

10. The device of claim 1, wherein the drug includes a biologic, and the second characteristic is an inflammatory response.

11. The device of claim 1, wherein the drug includes insulin, and the first characteristic is blood sugar level.

12. The device of claim 1, wherein the drug includes glucagon, and the first characteristic is blood sugar level.

13. The device of claim 1, wherein the drug includes a blood pressure medication, and the first characteristic is blood pressure.

14. The device of claim 1, wherein the at least one variable parameter includes a rate of delivery of the drug from the drug holder to the patient.

15. The device of claim 1, wherein the at least one variable parameter includes a time interval between dose deliveries such that doses delivered after the changing of the at least one variable parameter are at a different time interval than doses delivered before the changing of the at least one variable parameter.

16. The device of claim 1, wherein changing the at least one variable parameter results in the processor controlling delivery of the second dose such that the second dose is not delivered to the patient.

17. The device of claim 1, wherein the processor is configured to automatically change the at least one variable parameter.

18. The device of claim 1, wherein the processor is also configured to cause a notification to be provided to the patient based on the data gathered by the second sensor.

19. The device of claim 1, further comprising a communications interface configured to wirelessly transmit data indicative of the data gathered by the first sensor and data gathered by the second sensor to a remotely located computer system, and, in response, to wirelessly receive a command from the remotely located computer;
wherein the processor is configured to change the at least one variable parameter only after the communications interface receives the command.

20. The device of claim 1, wherein the processor is configured to change the at least one variable parameter of the algorithm during the delivery of the second dose such that the algorithm is changed in real time with the delivery of the second dose.

21. The device of claim 1, wherein the processor is configured to change the at least one variable parameter of the algorithm before a start of the delivery of the second dose.

22. The device of claim 1, wherein the memory is also configured to store therein manually input data regarding the patient; and
the processor is also configured to change the at least one variable parameter of the algorithm stored in the memory based on the stored input data.

23. The device of claim 1, wherein the drug comprises at least one of infliximab, golimumab, ustekinumab, daratumumab, guselkumab, epoetin alfa, risperidone, esketamine, ketamine, and paliperidone palmitate.

24-80. (canceled)
